(19) United States
(12) Patent Application Publication
(10) Pub. No.: US 2010/0016929 A1
(43) Pub. Date: Jan. 21, 2010

(54) METHOD AND SYSTEM FOR CONTROLLED NERVE ABLATION

(76) Inventor: Arthur Prochazka, Alberta (CA)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/565,377

(22) Filed: Sep. 23, 2009

Related U.S. Application Data
(63) Continuation-in-part of application No. 12/400,202, filed on Mar. 9, 2009.
Application No. 12/400,202 is a continuation of application No. 11/337,824, filed on Jan. 23, 2006, now Pat. No. 7,502,652.
Application No. 11/337,824 is a continuation-in-part of application No. PCT/CA2005/000074, filed on Jan. 24, 2005.
(60) Provisional application No. 60/538,618, filed on Jan. 22, 2004.

(51) Int. Cl. A61N 1/36

(52) U.S. Cl. 607/72

(57) ABSTRACT

The invention provides a system and method for treating a subject having unwanted or overactive nerve activity. The method involves applying one or more of direct current, charge imbalanced time varying current and pulsatile current to a target nerve; and controlling the amplitude and the duration of the current such that there is a net charge delivered to the target nerve at a sufficient charge density to cause controlled ablation to the target nerve until unwanted or overactive nerve activity is reduced in one or both of the target nerve and a target body tissue innervated by the target nerve.

### CROSS REFERENCE TO RELATED APPLICATIONS

This application is a Continuation-in-Part of pending U.S. patent application Ser. No. 12/400,202, filed Mar. 9, 2009, entitled “Method of Routing Electrical Current to Bodily Tissues Via Implanted Passive Conductors”, which is a Continuation of U.S. patent application Ser. No. 11/337,824, filed Jan. 23, 2006, entitled “Method of Routing Electrical Current to Bodily Tissues Via Implanted Passive Conductors”, which issued as U.S. Pat. No. 7,502,652 on Mar. 10, 2009, which is a Continuation-in-Part of International Application No. PCT/CA2005/000074 filed Jan. 24, 2005, which claims priority from U.S. Provisional Patent Application No. 60/538,618 filed Jan. 22, 2004. Each of the aforementioned applications is incorporated herein in its entirely by reference.

### BACKGROUND OF THE INVENTION

The present invention relates to an implant, system and method for treating a disorder of the nervous system in a subject. The method involves using passive electrical conductors which route electrical current to electrically stimulate a target body tissue to either activate or block neural impulses depending upon the form of the electrical current and the disorder to be treated.

Nerves consist of an axons that transmit action potentials to other excitable tissues such as neurons and muscle cells. In most cases the action potentials are transmitted to the terminals of the axons where neurotransmitters are released that act on receptors in the membranes of the receiving cells, initiating excitatory or inhibitory processes in these cells.

In some pathological states, transmission of action potentials is impaired; thus, activation of neural impulses by some other means can help to restore normal functioning. Electrically-excitable bodily tissues such as nerves and muscles may be activated by an electrical field applied between electrodes applied externally to the skin. Electric current flows through the skin between a cathode electrode and an anode electrode, eliciting action potentials in the nerves and muscles underlying the electrodes. This method has been used for many years in different types of stimulators, including transcutaneous electrical nerve stimulators (TENS) which relieve pain, therapeutic electrical stimulators which activate muscles for exercise purposes (Vodovnik, 1981), functional electrical stimulators which activate muscles for tasks of daily life (Kralj et al., 1989); U.S. Pat. No. 5,330,516 to Nathan; U.S. Pat. No. 5,562,707 to Prochazka et al.) and stimulators that promote regeneration of damaged bones.

In other pathological states, action potentials are transmitted which cause unwanted activity in the receiving cells, hence blocking of these action potentials can help restore normal functioning. It has been reported that high-frequency stimulation can produce temporary reversible blocks of nerve axons (Solomonow et al., 1983; Tai et al., 2004; Bhadra and Kilgore, 2005). Generally, the frequency range is between 500 and 30,000 Hz.

Stimulation of nerves to either active or block neural impulses is typically achieved with the use of an implanted stimulator (also known as a neural prosthesis or neuroprosthesis) (Peckham et al., 2001; Horch and Dhillon, 2004). Neural prostheses have been developed to restore hearing, to restore movement in paralyzed muscles, to modulate activity in nerves controlling urinary tract function and to suppress pain and tremor. In some cases, neural prostheses are designed to inhibit or suppress unwanted neural activity, for example to block pain sensation or tremors. However, all neural prostheses intended for long-term use require the implantation of a stimulator that contains electronic components and often a battery. In the case of pain and tremor suppression, the activated nerves reflexly inhibit the activity of neural circuits within the central nervous system. This indirect mode of reducing unwanted neural activity is sometimes called neuromodulation (Landau and Levy, 1993; Groen and Bosch, 2001).

Surface electrical stimulators are used reflexly for example to reduce spastic hypertonus (Vodovnik et al., 1984; Apkarian and Naumann, 1991). A disadvantage of stimulation through electrodes attached to the body surface is that many non-targeted tissues may be co-activated along with the targeted tissues. This lack of selectivity often causes unwanted sensations and/or unwanted movements. Furthermore, tissues that lie deep within the body are difficult or impossible to stimulate adequately, because most of the electrical current flowing between the electrodes flows through tissues closer to the electrodes than the targeted tissues. Selectivity may be improved by implanting wires within the body that route electrical current from a stimulator to the vicinity of the targeted tissues. This method is used in cardiac pacemakers (Horch et al., 2004), dorsal column stimulators (Waltz, 1997), deep brain stimulators (Benabid et al., 1987) and sacral root stimulators (Brindley et al., 1982). Cuffs containing the uninsulated ends of the wires may be placed around peripheral nerves to restrict most of the current to the vicinity of the nerve and limiting the spread of current to surrounding tissues, thereby improving selectivity (Haugland et al., 1999). Generally when wires are implanted, the stimulators, complete with an energy source, are also implanted (Strojnik et al., 1987). Implanted stimulators are expensive and often require a controller and/or power source external to the body. Batteries within the implanted stimulators need periodic replacement, entailing surgery.

In a minority of cases, stimulating wires are implanted in bodily tissues and led through the skin (percutaneously) to a connector attached to the surface of the body, to which an external stimulator is attached (Peckham et al., 1980; Handa et al., 1998; Shaker and Hassouna, 1999; Yu et al., 2001). External stimulators are much less expensive than implanted stimulators, but the percutaneous wires provide a conduit for infection and therefore require daily cleaning and maintenance. This has generally limited the use of percutaneous electrodes to short-term applications. There is a need for a system which overcomes such problems and has the capability of activating or blocking nerve impulses depending upon the disorder to be treated.

### Overactivity or Unwanted Activity of Peripheral Nerves

In disorders such as stroke, multiple sclerosis, cerebral palsy and spinal cord injury (SCI), overactivity of both the sensory and motor components of nerves innervating bodily tissues such as the muscles that control movements of the limbs, trunk and head and autonomic functions such as bladder and sphincter contractions, can be seriously disabling. Over 80% of people with multiple sclerosis (MS) have been reported to suffer from spastic hypertonus, which is caused by overactivity in nerves innervating muscles and which can cause muscle stiffness and painful, disruptive spasms. A survey of people living with SCI revealed that spastic hypertonus developed in 65% of cases (Skold et al., 1999). A smaller proportion of stroke survivors develop spasticity, around 20%, but this still amounts to nearly 1 million people in North America alone. Some movement disorders such as the dystonias also involve tonic overactivity of muscles. Overactivity of sensory nerves can cause pain and burning sensations, parasthesiae and hyperreflexia.

In addition to these clinical disorders, involuntary activity of certain muscles in neurologically normal individuals, for example muscles of the forehead and around the eyes, lead to skin wrinkles which can be reduced by inactivating the nerves to these muscles.

### (i) Current Clinical Treatments

Current physiotherapeutic methods to treat spasticity include muscle stretching, exercise, brushing, vibration, casting, pressure splints and transcutaneous electrical stimulation. The efficacy of these treatments is often quite limited, variable and of short duration. Commonly used antispastic drugs include those acting centrally such as baclofen, diazepam, tizanidine and clonidine and those acting on the neuromuscular junction, such as dantrolene. These all have moderate to severe side effects, including fatigue, weakness and cognitive effects such as drowsiness and sedation. The majority of people living with chronic stroke, SCI and MS discontinue anti-spastic medication after some time. A more targeted, albeit expensive approach is the intrathecal release of drugs such as baclofen by means of a programmable implanted mini-pump. Another fairly common pharmacological treatment involves the localized injection of agents that block nerve conduction such as phenol (Kirazli et al. 1998) and more recently, botulinum toxin A (BtA) (Ade-Hall & Moore 2000). Finally, surgical interventions such as tendon lengthening, joint fusion and osteotomy provide further options in severe cases (Woo R. 2001).

Phenol and alcohol injections have been used since the 1950s to reduce spastic hypertonus and dystonia (McCrea et al. 2004). A fairly frequent side effect of phenol blocks is the occurrence of dysesthesias (uncomfortable sensations such as burning, itching and shooting pain), which may last for several weeks. Skill is required to deliver the drug to the targeted nerves and not all clinical centers have personnel with the appropriate training and experience to perform this procedure. Injected fluids generally do not stay in localized globules but rather they slip along muscle and connective tissue planes (Amis et al. 1987). This may explain the variability of functional outcomes and the occurrence of pain in tissues adjacent to the targeted nerves.

A recent randomized controlled trial slightly favored BtA over phenol (Kirazli et al. 1998). However, because of the high incidence of painful side-effects of phenol, BtA has become the more popular nerve-blocking treatment for spasticity in most centers. BtA acts primarily at neuromuscular junctions, which are the terminals of the large axons in nerves that transmit motor commands to muscles. Neuromuscular junctions tend to be clustered in groups at several sites in muscles, corresponding to the terminal branches of the nerves. These sites can be quite widely spaced, so to achieve an adequate motor point block, BtA is usually injected at several locations within muscles, and in several muscles. Unlike phenol or alcohol, whose blocking effects are immediate, BtA takes up to two weeks to act. This means the efficacy of the injections, which depends on the choice of injection sites and dosage, depends on the clinician's skill and experience rather than immediate feedback. Mild-flu-like symptoms can occur for a week or two after BtA injections. The most important limitation of BtA however is the relatively short duration of efficacy, typically 4 months, necessitating repeated sets of expensive injections that are not always covered by third party payors. A recent study in the UK concluded that it costs the healthcare system between US$20,000 and US$40,000 per year per person to provide continuous anti-spasticity treatment using BtA or oral drugs (Ward et al. 2005).

### (ii) Surgical Interventions

Tendon release surgery is most often used to alleviate severe muscle contractures resulting from hypertonus in cerebral palsy. Multi-site tendon surgery can improve gait by reducing contractures of muscles acting about the hip, knee, and ankle. One of the main problems with tendon release surgery is the relatively long period of post-operative immobilization required, followed by aggressive exercise training. Selective dorsal rhizotomy is another fairly common surgical procedure to avoid or reduce spastic contractures in cerebral palsy. Partial transection of peripheral nerves is also performed in some cosmetic surgical procedures, for example to reduce unwanted muscle bulk in procedures such as calf reduction.

The above surgical procedures can be very effective, but the costs, including those associated with post-operative care, rehabilitation and repeated treatments are high compared to oral or nerve-blocking drug treatments.

### Electrical Blockade of Nerves

Action potential propagation in the axons of peripheral nerves may be blocked either with high-frequency alternating current (HFAC) or direct current (DC). HFAC blockade was discovered in 1935 (Cattell M & Gerard R W. 1935). Since then it has been investigated sporadically, but recently there has been renewed interest in its potential clinical applications, e.g. pudendal nerve blockade to counteract bladder-sphincter dyssynergia (Tai et al. 2007). The mechanism of HFAC blockade is not well understood, nor are the factors that determine the completeness of blockade, undesirable side-effects such as onset transients and tissue damage, and the speed of post-blockade recovery.

With respect to DC blockade, in studies of nociceptive (pain) transmission in peripheral nerves it was found that large diameter nerve axons that mediate sensations such as touch, pressure and movement can be selectively blocked with DC lasting some minutes (Whitwam & Kidd 1975). It was found necessary to gradually reduce the current from an initial level, otherwise the smaller afferents that mediate nociception became blocked too. The authors concluded that “The damage which repeated application of electrical current causes in a nerve renders this technique unsuitable for clinical use, where complete recovery is essential.” Thus these authors taught away from the use of DC blockade as a clinical treatment. It should be noted that the data were obtained in experiments lasting less than 24 hours in anesthetized animals and histological analysis was not performed. There was no intent to perform this procedure in the absence of anesthesia, or for long-term nerve block.

Implanted neuroprostheses are generally designed to activate the axons within nerves rather than to block propagation of action potentials in them. Charge-balanced biphasic pulses with durations in the range 0.05 to 0.5 ms or bursts of alternating current are generally delivered by neuroprostheses to activate nerves. Much work has been done to identify the parameters of pulsatile stimulation that are either “safe” or “unsafe.” The relevant parameters are pulse duration, amplitude and rate, percentage of charge retrieval in biphasic pulses, charge density and charge per phase (McCreery, 1992). Because nearly all of this work has been directed at ensuring that neuroprostheses do not cause neural damage, effort has been directed at determining the safe stimulation parameters, which teaches away from utilizing the unsafe region. Short pulse durations avoid irreversible electrochemical reactions that may damage axons (McCreery et al. (1990). The question arises, is there a specific duration beyond which an applied current should not be referred to as a pulse, but rather as DC stimulation? In their review of the literature, Bhadra & Kilgore (2004) do not specify such a duration, but most of the papers to which they refer involve durations ranging from 1 second to several minutes. In what follows we will therefore take 1 second as the dividing line between pulsatile and DC stimulation.

DC combined with HFAC blockade is suggested by Kilgore and Bhadra (WO 2009/058258). They propose the use of a short period of DC stimulation prior to the onset of HFAC stimulation to block nerves. The function of the DC stimulation in this case is temporarily to block the nerve so as to avoid the transient activation of axons, including those mediating pain, that is associated with the onset of HFAC blockade. In their method, Kilgore and Bhadra (20009) do not intend that the DC stimulation be the primary method of nerve blockade, nor do they teach the use of either DC or HFAC to block the nerve permanently or for a long period by damaging its axons intentionally. In fact Kilgore and Bhadra (2009) teach away from the use of damaging parameters of stimulation such as long-duration DC or charge-imbalanced HFAC by proposing various waveforms and durations of DC and HFAC stimulation to minimize charge imbalance and thereby to avoid damaging the nerve.

Electrical stimulation at RF frequencies has been used to ablate neural tissue. For example, destructive electrical stimulation applied through microelectrodes has provided a valuable means of marking microelectrode recording sites. Ablation of specific brain areas has also been performed for many years in the treatment of Parkinson's disease and essential tremor. This is referred to as electrocoagulation, which is usually performed with radio-frequency current at frequencies greater than 80 KHz (Jankovic et al. 1995). Heggeness (U.S. Pat. No. 6,699,242) describes the use of electrical current delivered by a surgical probe to ablate nerves within vertebral bones of the spinal column. The probe is specifically designed to penetrate bone. No details of the characteristics of the electrical current are given. The method is to insert the probe into the bone, deliver the ablative electrical current and then to remove the probe.

### Mechanisms of Nerve Damage

The mechanisms for stimulation-induced tissue damage are not well understood. Scheiner et al. (1990) applied large imbalanced biphasic current pulses via intramuscular electrodes and subsequently found histological evidence of coagulated, necrotic axons and muscle fibers. The mechanisms of damage proposed included heating, direct electric field effects and loss of blood flow. Experiments in a frog nerve-muscle preparation indicated that DC causes a depolarization block, resulting in the closing of the inactivation gates in sodium channels in the axonal cell membrane under the cathodal electrode (Bhadra & Kilgore 2004). Several other possibilities have been proposed in the literature. For example, irreversible electrochemical reactions that occur at “unsafe” stimulation levels result in changes in pH that can alter cellular proteins. The evolution of oxygen and hydrogen gas and reactive oxygen species like superoxide and hydrogen peroxide can cause demyelination and may disrupt nitric oxide synthesis, thus inhibiting vasodilation and decreasing perfusion. Corrosion and dissolution of the metal electrodes into neural and surrounding tissues can occur, further contributing to tissue damage. Another possible mechanism for nerve damage is a phenomenon known as “mass action,” the result of hyperactivity and overstimulation of nerves (Merrill et al. 2005).

### SUMMARY

The present invention broadly relates to an implant, system and method using passive electrical conductors which route electrical current to electrically stimulate a target body tissue to either activate or block neural impulses depending upon the frequency and the disorder to be treated.

In one aspect, the present invention broadly provides an implant for electrically stimulating a target body tissue in a subject, the implant, once implanted, providing a conductive pathway for at least a portion of the electrical current flowing between surface cathodic and anodic electrodes positioned in spaced relationship on the subject's skin and transmitting that portion of the electrical current to the target body tissue, the implant comprising:

a passive electrical conductor of sufficient length to extend, once implanted, from subcutaneous tissue located below the surface cathodic electrode to the target body tissue, the electrical conductor having a pick-up end and a stimulating end and being insulated between its ends, the pick-up end forming an electrical termination having a sufficient surface area to allow a sufficient portion of the electrical current to flow through the conductor, in preference to flowing through body tissue between the surface cathodic and anodic electrodes, such that the target body tissue is stimulated, and the stimulating end forming an electrical termination for delivering the portion of electrical current to the target body tissue.

In another aspect, the invention provides a system for electrically stimulating a target body tissue in a subject comprising the above implant, together with

surface cathodic and anodic electrodes for making electrical contact with the subject's skin, and which, when positioned in spaced relationship on the subject's skin, for transmitting electrical current to the target body tissue; and

stimulator external to the subject's body, electrically connected to the surface cathodic and anodic electrodes, the stimulator supplying direct, pulsatile, or alternating current to the surface cathodic and anodic electrodes.

In another aspect, the invention provides a method for electrically stimulating a target body tissue in a subject comprising the steps of:

providing the above implant;

implanting the implant entirely under the subject's skin, with the pick-up end positioned in subcutaneous tissue located below the surface cathodic electrode, and the stimulating end positioned proximate to the target body tissue;

positioning the surface cathodic and anodic electrodes in spaced relationship on the subject's skin, with the surface cathodic electrode positioned over the pick-up end of the electrical conductor so the portion of the current is transmitted through the conductor to the target body tissue, and so that the current flows through the target body tissue and returns to the anodic surface electrode through body tissues or through an implanted electrical return conductor extending between the target body tissue and subcutaneous tissue located below the surface anodic electrode; and

applying direct, pulsatile or alternating electrical current between the surface cathodic electrode and the surface anodic electrode to cause the portion of the electrical current to flow through the implant sufficient to stimulate the target body tissue.

In yet another aspect, the present invention provides a method of treating a disorder in a subject comprising the steps of:

providing an implant to act as a conductive pathway for at least a portion of the electrical current flowing between surface cathodic and anodic electrodes positioned in spaced relationship on the subject's skin and transmitting the portion of the electrical current to the target body tissue, the implant comprising a passive electrical conductor of sufficient length to extend, once implanted, from subcutaneous tissue located below the surface cathodic electrode to the target body tissue, the electrical conductor having a pick-up end and a stimulating end and being insulated between its ends, the pick-up end forming an electrical termination having a sufficient surface area to allow a sufficient portion of the electrical current to flow through the conductor, in preference to flowing through body tissue between the surface cathodic and anodic electrodes, such that the target body tissue is blocked, and the stimulating end forming an electrical termination for delivering the portion of electrical current to the target body tissue;

implanting the implant entirely under the subject's skin, with the pick-up end positioned in subcutaneous tissue located below the surface cathodic electrode, and the stimulating end positioned proximate to the target body tissue;

positioning the surface cathodic and anodic electrodes in spaced relationship on the subject's skin, with the surface cathodic electrode positioned over the pick-up end of the electrical conductor so the portion of the current is transmitted through the conductor to the target body tissue, and so that the current flows through the target body tissue and returns to the anodic surface electrode through body tissues or through an implanted electrical return conductor extending between the target body tissue and subcutaneous tissue located below the surface anodic electrode; and

applying electrical current between the surface cathodic electrode and the surface anodic electrode in the form of a cyclical waveform at a frequency capable of blocking the target body tissue so as to treat the disorder.

The invention also broadly provides a method of treating a subject having unwanted or overactive nerve activity, comprising:
(a) applying one or more of direct current and charge imbalanced time varying current to a target nerve; and
(b) controlling the amplitude and the duration of the current such that there is a net charge delivered to the target nerve at a sufficient current density to cause controlled ablation of the target nerve until unwanted or overactive nerve activity is reduced in one or both of the target nerve and a target body tissue innervated by the target nerve.

The method is preferably conducted while the subject is sedated or anaesthetized. However, within the parameters of controlled ablation, the method has been found to produce no signs of fasciculation or pain or discomfort after recovery from anesthesia. Thus, although the method provides reversible or permanent nerve blockade through nerve ablation, including destruction of motor and sensory axons, none were left in a spontaneously active state, as can occur after injections of phenol. The method also shows no evidence of dysesthesia.

Preferred parameters for the method include the application of direct current delivered to the target nerve at a current density of between 0.2 mA/cm² and 12 mA/cm². In some embodiments, the application of direct current delivered to the target nerve is at a current density of between 0.3 mA/cm² and 4 mA/cm². In some embodiments, the application of direct current delivered to the target nerve is at a current density of between 0.4 mA/cm² and 2 mA/cm². The method preferably includes applying current for a duration of more 1 second, or more than 10 seconds, or more than one minute or more than 10 minutes, or more than one hour or more than a day. The duration will vary with such factors as current amplitude, charge density, size of electrical terminations at a delivery end of an implant delivering the current, and the proximity of the delivery end to the target nerve. The current may be applied continuously or intermittently. Alternatively, the current may be delivered as charge imbalanced time varying current.

The method is particularly preferred for application to peripheral nerves, including without limitation a facial nerve, a spinal accessory nerve, a musculocutaneous nerve, a median nerve, a pudendal nerve, a sciatic nerve, a femoral nerve, and one or more branches of one of these nerves.

The method is preferably practiced using one or more implanted conductors having a delivery portion positioned proximate to, or attached to, the target nerve. The current source may be external or implanted. Once the implanted conductor is in place, the method has the advantage of allowing for re-application of the current when the unwanted or overactive nerve activity returns, with the re-application being a simple procedure.

In a preferred embodiment, the method is practiced with a fully implanted implant. The method comprises:
(a) implanting an implant under the subject's skin, the implant including a passive electrical conductor having a pick-up portion and a delivery portion and being insulated between the pick-up portion and the delivery portion, the pick-up portion being configured to pick up at least a portion of a current flowing between a first surface electrode and a second surface electrode when positioned on the subject's skin, and to transmit the portion of the current to a target nerve;
(b) positioning the first surface electrode and the second surface electrode in spaced relationship on the subject's skin to make direct electrical contact with the subject's skin, with the first surface electrode positioned over the pick-up portion of the electrical conductor so the portion of the current is transmitted through the electrical conductor to the target nerve; and
(c) applying one or more of direct current and charge imbalanced time varying current between the first surface electrode and the second surface electrode to cause the portion of the current to flow through the implant to be delivered to the target nerve; and
(d) controlling the amplitude and the duration of the electrical current such that there is a net charge delivered to the target nerve at a sufficient current density to cause controlled ablation of the target nerve until unwanted or overactive nerve activity is reduced in one or both of the target nerve and a target body tissue innervated by the target nerve.

The invention also extends to a system for practicing the present invention.

As used herein and in the claims, the terms and phrases set out below have the following definitions.

“Blocking” or “block” is meant to refer to preventing the conduction or propagation of action potentials or nerve impulses along the axons of a target nerve partially or completely.

“Body tissue” is meant to refer to a neural tissue (in the peripheral or central nervous system), a nerve, a muscle (skeletal, respiratory, or cardiac muscle) or an organ, for example, the brain, cochlea, optic nerve, heart, bladder, urethra, kidneys and bones.

“Charge imbalanced pulsatile current” means pulsatile current delivered in a manner such that a net charge is delivered to a target nerve.

“Charge imbalanced time varying current” means current delivered in a time varying manner such that a net charge is delivered to a target nerve.

“Cyclical waveform” means any form of electrical current in a repeating waveform without limitation to its shape or form, including without limitation alternating current, pulsatile, sinusoidal, triangular, rectangular and sawtooth waveforms.

“Direct current” is meant to include continuous direct current and pulsatile current with pulses lasting at least as long as one second.

“Disorder” is meant to include movement disorders, muscular disorders, incontinence, urinary retention, pain, epilepsy, cerebrovascular disorders, sleep disorders, autonomic disorders, disorders of vision, hearing and balance, and neuropsychiatric disorders.

“Electrical current” is meant to refer to current applied at the surface of the skin that is resistively and capacitively coupled to the implanted passive conductor, which in turn conveys the current to the target neural tissue.

“Nerve ablation” and “nerve lesioning” mean the destruction of one or more axons of a target nerve so as to result in a nerve blockade in which conduction or propagation of action potentials in the target nerve is attenuated or abolished, either reversibly or permanently, as evidenced by the attenuation or abolition of sensation normally mediated by the nerve or weakness or paralysis of the body tissue innervated by the target nerve lasting more than a week, more than two weeks or more than a month.

“Subject” means an animal including a human.

### BRIEF DESCRIPTION OF THE DRAWINGS

FIG. 1 is a schematic three-dimensional view of an embodiment of the invention having an implanted electrical conductor, surface cathodic and anodic electrodes, and an implanted electrical return conductor.

FIG. 2 is a side elevation view, in section, of an embodiment of the invention having an implanted electrical conductor and surface cathodic and anodic electrodes.

FIG. 3 is a side elevation view, in section, of an alternate embodiment of the invention having an implanted electrical conductor, surface cathodic and anodic electrodes, and an electrical return conductor.

FIG. 4 is a side elevation view, in section, of an alternate embodiment of the invention having two implanted electrical conductors, two surface cathodic electrodes, an anodic electrode, and an electrical return conductor.

FIGS. 5A and 5B are graphs showing the effect of frequency and amplitude on pudendal nerve blocking. FIG. 5A shows the maximum decrease in urethral pressure elicited by stimulation of the pudendal nerve at different amplitudes and frequencies, with the maximum decrease defined as the difference between the intraurethral pressure just prior to and during high frequency stimulation. FIG. 5B shows the difference between background intraurethral pressure and the intraurethral pressure obtained during high frequency stimulation at different amplitudes and frequencies.

FIGS. 6A and 6B are graphs showing the effect of stimulation amplitudes of 1 mA (FIG. 6A) and 3 mA (FIG. 6B) with a frequency of 1 kHz on pudendal nerve blocking in one animal.

FIGS. 7A and 7B are graphs showing the effect of stimulation amplitudes of 6 mA (FIG. 7A) and 3 mA (FIG. 7B) with a frequency of 2 kHz on pudendal nerve blocking in one animal.

FIG. 8 is a graph showing the relationship between urethral pressure and bladder pressure during pudendal nerve blocking in one animal.

FIG. 9 is a schematic view, in section, of a system and method of delivering current to a target nerve in order to cause controlled nerve ablation, using surface electrodes and a single implanted conductor.

FIG. 10 is a schematic view, in section, of a system and method of delivering current to a target nerve in order to cause controlled nerve ablation, using surface electrodes and two implanted conductors.

FIG. 11 is a schematic side elevation view, in section, of a system and method of delivering current to a target nerve in order to cause nerve ablation, using an external inductively coupled controller, an implanted current source and a single implanted conductor.

FIG. 12 is a schematic view, in section, of a system and method of delivering current to a target nerve in order to cause nerve ablation, using an external inductively coupled controller, an implanted current source and two implanted conductors.

### DETAILED DESCRIPTION

The invention broadly provides an implant for electrically stimulating a target body tissue in a subject to either activate or block neural impulses depending upon the frequency and the disorder to be treated. Once implanted, the implant provides a conductive pathway for at least a portion of the electrical current flowing between surface cathodic and anodic electrodes positioned in spaced relationship on a subject's skin, and transmits that portion of electrical current to the target body tissue to either activate or block neural impulses. In further aspects, the invention provides a system and method incorporating the implant.

The subject can be an animal including a human. The body tissue can be a neural tissue (in the peripheral or central nervous system), a nerve, a muscle (skeletal, respiratory, or cardiac muscle) or an organ, for example, the brain, cochlea, optic nerve, heart, bladder, urethra, kidneys and bones.

The invention can be applied to treat various conditions in which stimulation to either activate or block neural impulses is required. Such conditions can include movement disorders (e.g., spasticity, hypertonus, rigidity, tremor and/or muscle weakness, Parkinson's disease, dystonia, cerebral palsy), muscular disorders (e.g., muscular dystrophy), incontinence (e.g., urinary bladder disorders), urinary retention, pain (e.g., migraine headaches, neck and back pain, pain resulting from other medical conditions), epilepsy (e.g., generalized and partial seizure disorder), cerebrovascular disorders (e.g., strokes, aneurysms), sleep disorders (e.g., sleep apnea), autonomic disorders (e.g., gastrointestinal disorders, cardiovascular disorders), disorders of vision, hearing and balance, and neuropsychiatric disorders (e.g., depression). The invention may also be used for promoting bone growth (as required, for example, in the healing of a fracture), wound healing or tissue regeneration.

For stimulation of a target body tissue, particular frequencies to be applied depend upon many factors; for example, the type of nerve to be blocked, the tissue which the nerve innervates, the size of the nerve, the subject to be treated, the type of condition, the severity of the condition, and the receptiveness of the subject to the treatment. In general, for blocking, high frequencies are useful, for example, the cyclical waveform can be applied at a frequency in the range of between 100 and 30,000 Hz, or alternatively in the range of between 100 and 20,000 Hz. Still alternatively, the cyclical waveform can be applied at a frequency in the range of between 100 and 10,000 Hz, or in the range between 200 and 5,000 Hz. For activation, low frequencies are generally used, for example, a frequency in the range of between 1 and 100 Hz, or alternatively, in the range of between 1 and 50 Hz. Still alternatively, the frequency can be in the range of between 1 and 20 Hz.

### A. The Router System

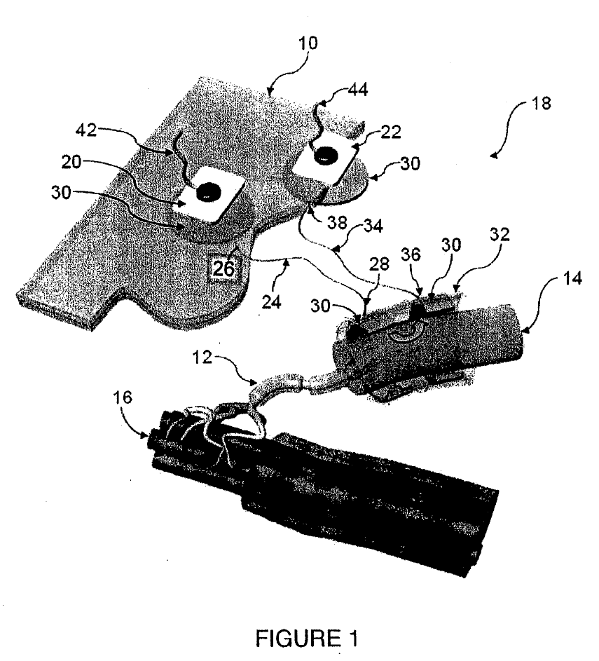

The invention is described with reference to the drawings in which like parts are labeled with the same numbers in FIGS. 1 to 4. The invention is shown generally in FIG. 1 which schematically illustrates portions of a subject's body tissues, including skin 10, a nerve 12 with its overlying nerve sheath 14, and a muscle 16. FIG. 1 also illustrates an implant indicated generally at 18, a surface cathodic electrode 20 and a surface anodic electrode 22. The implant 18 is provided for electrically stimulating a target body tissue, such as a nerve 12, in a subject to either activate or block neural impulses. Once implanted, the implant 18 provides a conductive pathway for at least a portion of the electrical current flowing between the surface cathodic and anodic electrodes 20, 22.

When positioned in spaced relationship on the subject's skin 10, the surface cathodic and anodic electrodes 20, 22 make electrical contact with the skin 10 and transmit electrical current to the target body tissue. Surface cathodic and anodic electrodes 20, 22 can be selected from a conductive plate or sheet, a conductive gel electrode, a conductive rubber or polymer electrode that may be partially coated with an electrode paste or gel, or a moistened absorbent pad electrode. Self-adhesive hydrogel electrodes of the type used to stimulate muscles, with surface areas of 1 square centimeter or more are particularly effective. Platinum iridium electrodes, which are composed typically of 80% or more platinum and 20% or less iridium, can also be used (for example, 85% platinum-15% iridium alloy; 90% platinum-10% iridium alloy). The positions of the surface cathodic and anodic electrodes 20, 22 on the skin 10 may vary, depending upon the location and nature of the target body tissue.

A plurality of surface electrodes 20, 22 may be fabricated on a single non-conductive substrate to form an electrode array that may be conveniently attached to the skin 10 in one maneuver. Similarly, the plurality of terminations 30 of implanted conductors 24 may be fabricated on a substrate to form an array. By matching the physical layout of the surface electrode array to that of the implanted terminations array, a good spatial correspondence of surface and implanted conductors may be achieved in a convenient and reproducible manner. Surface electrode arrays in which the conductivity of each element of the array may be independently controlled could also be used to adjust the conductivity between the surface electrodes and the terminations in an implanted array.

The implant 18 comprises a passive electrical conductor 24 of sufficient length to extend, once implanted, from subcutaneous tissue located below the surface cathodic electrode 20 to the target body tissue, for example nerve 12. The electrical conductor 24 can be formed from a metal wire, carbon fibers, a conductive rubber or other conductive polymer, or a conductive salt solution in rubber. Multistranded, TEFLON®-insulated, stainless-steel wire conductors of the type used in cardiac pacemaker leads have been found to be particularly effective. MP35N® alloy (a nonmagnetic, nickel-cobalt-chromium-molybdenum alloy) which is commonly used in parts for medical applications is also suitable. The electrical conductor has a pick-up end 26 and a stimulating end 28, and is insulated between its ends 26, 28.

The electrical impedance of the interface between the ends 26, 28 of the conductor 24 (when implanted) and the surrounding body tissue may be reduced by enlarging the surface area of the ends 26, 28. For that purpose, one or both of the pick-up and stimulating ends 26, 28 form electrical terminations 30 having sufficient surface areas for reducing the electrical impedance of the interface between the pick-up and stimulating ends 26, 28 of the electrical conductor 24 and the surrounding body tissues. Preferably, the pick-up end 26 forms a termination 30. The pick-up end 26 forms an electrical termination 30 which has a sufficient surface area to allow a sufficient portion of the electrical current to flow through the electrical conductor 24, in preference to flowing through body tissue between the surface cathodic and anodic electrodes 20, 22, such that the target body tissue is stimulated to either activate or block neural impulses. The stimulating end 28 also forms an electrical termination 30 for delivering the portion of electrical current to the target body tissue (i.e., nerve 12).

Terminations 30 should have sufficient surface area for providing high conductivity contact with body tissues, and lowering the electrical impedance between the body tissue and the conductor. If the surface area is minimal, the amount of current flowing through a conductor to the termination is reduced to an ineffective amount. The surface area required may thus be determined by a knowledge of the electrical impedance of the interface between the tissue and the terminations 30 at the receiving and stimulating ends 26, 28. Beneficial results have been obtained by making the surface area of metal terminations 30 at the ends 26, 28 about 0.5 cm². The electrical impedance of each interface between tissue and terminations 30 at ends 26, 28 was then about 5 times the electrical impedance of all the subcutaneous tissue between surface electrodes 20, 22. A typical value of tissue impedance is 200 ohms. The impedance of the conductor itself is chosen to be very small, for example 5 ohms. In the example just given, the sum of the two interface impedances of the terminations 30 plus the conductor impedance was about 2000 ohms, that is to say about ten times the tissue impedance. Thus about 10% of the current applied between surface electrodes 20, 22 flows through conductor 24 to the target tissue. In the case of the target tissue being a nerve 12 supplying a muscle 16, the amount of current between surface electrodes 20, 22 required to produce a useful muscle contraction of the target muscle 16 then remains below the threshold level of activation of nerve endings in the subcutaneous tissue immediately between surface electrodes 20, 22. This is a beneficial relationship, because it means that target muscles 16 can be activated with little or no local sensation under the surface electrodes 20, 22.

Terminations 30 of various shapes, materials and spatial arrangements can be used; for example, terminations 30 can provide an enlarged surface in the form of a coil, spiral, cuff, rod, or a plate or sheet in the form of an oval or polygon. As an example, FIG. 1 illustrates a termination 30 as a plate or sheet in the form of an oval at the pick-up end 26 of the electrical conductor 24, and in the form of a cuff at the stimulating end 28. The cuff or a portion thereof can encircle or partially encircle the entirety or part of the nerve sheath 14 of the nerve 12. The cuff or a portion thereof can be positioned proximate to the nerve sheath 14, or the inner surface of the cuff or a portion thereof can directly contact the nerve sheath 14.

Beneficial results are obtained with stainless-steel plates or sheets in the form of an oval which is about 0.5 cm² in area and 1 mm thick, or made of metal foil and stainless-steel mesh and being about 0.5 cm² in surface area and 0.3 mm thick. For terminations 30 of conductors with nerve cuffs, nerve cuffs made of metal foil or stainless-steel mesh and being 0.5 to 1 cm² in surface area and 0.3 mm thick are suitable. Further, silastic elastomer cuffs ranging from 5 mm to 15 mm in length, 4 mm to 6 mm inside diameter, and 1 mm thick are suitable.

Terminations 30 can be formed from uninsulated ends 26, 28 of the electrical conductor 24, or from other conductive or capacitive materials. Terminations 30 can be formed by coiling, spiraling or weaving long, uninsulated lengths of the pick-up or stimulating ends 26, 28 to provide a sufficient surface. The surface area of the termination is thus “enlarged” relative to the surface area of a shorter length of the electrical conductor 24. This raises the effective surface area of the terminations 30 within a small space to provide higher conductivity contact with body tissues, and to lower the electrical impedance between the body tissue and the conductor 24 to allow current flow in the conductor in preference to in the body tissue. Sufficient current flow is thereby provided in the conductor 24 to stimulate the target tissue. Alternatively, prefabricated terminations 30 (for example, plates or sheets in the form of ovals or polygons) can be attached directly to the pick-up and stimulating ends 26, 28. Further, terminations 30 can be coated or modified with conductive materials to maximize the flow of electrical current through the target body tissue.

The spatial arrangement of the terminations 30 can be varied; for example, multiple terminations 30 can also be applied to different parts of a body tissue (Grill et al., 1996). Advantageously, the terminations 30 themselves can be in the form of closely-spaced contacts enclosed within an embracing cuff 32 placed around the nerve 12. The embracing cuff 32 can be formed from conductive silicone rubber.

Electrical impedance may be further reduced by providing conductive or capacitive coatings, or an oxide layer on the terminations 30. The coating can be selected from a material whose structural or electrical properties improve the electrical conductance between the tissue and the conductor, for example, by providing a complex surface into which tissue can grow (for example, a polymer such as poly-diethoxy-thiophene, or suitable oxide layers including tantalum and sintered iridium). In addition, the terminations 30 can have coatings which provide an anti-inflammatory, anti-bacterial or tissue ingrowth effect. The coating can be a substance selected from an anti-inflammatory agent, antibacterial agent, antibiotic, or a tissue ingrowth promoter.

Optionally, performance of the invention can be improved by implanting an electrical return conductor 34 of sufficient length to extend from the target body tissue to subcutaneous tissue located below the surface anodic electrode 22. The electrical return conductor 34 provides a low-impedance conductive pathway from the target body tissue to the surface anodic electrode 22, thereby concentrating the electric field through the target tissue. The electrical return conductor 34 can be formed from a metal wire, carbon fibers, a conductive rubber or other conductive polymer, or a conductive salt solution in rubber. The electrical return conductor 34 has a collecting end 36 and a returning end 38, and is insulated between its ends 36, 38. Both the collecting end 36 and the returning end 38 form electrical terminations 30 (as described above) for reducing the electrical impedance of the interface between the collecting end 36 and returning end 38 of the electrical return conductor 34 and the surrounding body tissues. The collecting end 36 forms an electrical termination 30 (shown in FIG. 1 in the form of a cuff), which has a sufficient surface area to allow a portion of the electrical current delivered to the target body tissue to return through the electrical return conductor 34 in preference to returning through body tissue. The returning end 38 forms an electrical termination 30 (shown in FIG. 1 as a plate or sheet in the form of an oval) which returns the electrical current to the surface anodic electrode 22 via the subcutaneous tissue and skin underlying the surface anodic electrode 22.

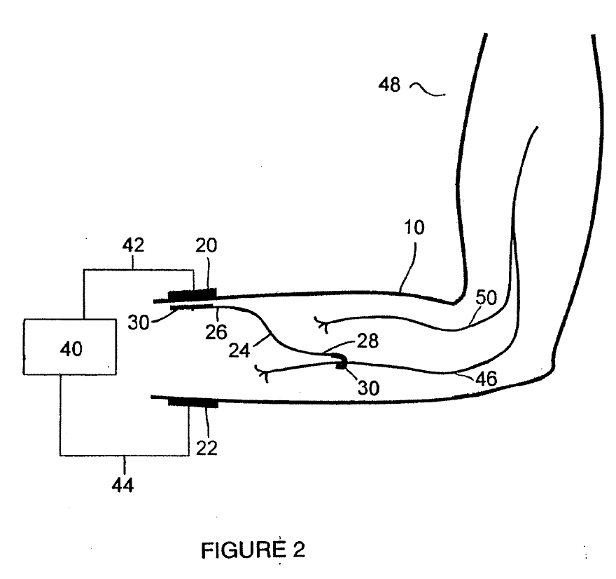

A power source 40 (shown in FIGS. 2-4) provides operating power to a stimulator (not illustrated) which is external to the subject's body. The stimulator is electrically connected to the surface cathodic and anodic electrodes 20, 22 to supply electrical current to the surface cathodic and anodic electrodes 20, 22. The current can be resistive or capacitive, depending on the net impedance encountered between the electrodes 20, 22.

Although most of the electrical current flows through the body tissues in proximity to the surface cathodic and anodic electrodes 20, 22, there is flow of electrical current through the electrical conductor 24, nerve 12, and electrical return conductor 34. As shown in FIG. 1, the surface cathodic electrode 20 is positioned over the pick-up end 26 of the electrical conductor 24, so that a portion of the current is transmitted through the conductor 24 to the target body tissue, and current flows through the target body tissue and returns to the anodic surface electrode 22 through body tissues. This can also be achieved through the implanted electrical return conductor 34 extending between the target body tissue and subcutaneous tissue located below the surface anodic electrode 22.

The complete electrical path of the portion of the electrical current is as follows: cathodic wire 42, surface cathodic electrode 20, skin 10, termination 30, pick-up end 26, electrical conductor 24, stimulating end 28, termination 30, nerve sheath 14, nerve 12, termination 30, collecting end 36, electrical return conductor 34, returning end 38, termination 30, skin 10, surface anodic electrode 22 and anodic wire 44. The pulses of electrical current can elicit action potentials which are conducted along nerve 12 to muscle 16, causing it to contract. Alternatively, electrical current in the form of high frequency waveforms can block action potentials conducted along nerve 12 to muscle 16 to prevent muscle contractions.

Various disorders are amenable to treatment by the invention as shown in FIG. 1. As described below, the implanted passive electrical conductors of the present invention are capable of routing electrical current to stimulate various target body tissues to either activate or block neural impulses depending upon the frequency and disorder to be treated. Applications have been provided below to illustrate examples of target body tissues and disorders for which the invention is beneficial.

### B. Activation of Neural Impulses Using the Router System

In some pathological states, transmission of action potentials is impaired; thus, activation of neural impulses is required to restore normal functioning. In the present invention, the stimulator can supply direct, pulsatile or alternating current between the surface cathodic and anodic electrodes 20, 22 to cause the portion of the electrical current to flow through the implant 18 sufficient to stimulate the target body tissue to activate neural impulses.

Exemplary pulse parameters of electrical current flowing between the surface cathodic and anodic electrodes 20, 22 for activation of neural impulses are as follows: biphasic current pulses, 30 pulses per second, each phase 200 microseconds in duration, and a peak current per pulse ranging from 0.7 to 2 milliampere. Beneficial results can be obtained with rectangular, feedback-controlled current pulse waveforms, although other waveforms and modes of control of current or voltage have also been found to give satisfactory results. The inventor has discovered that between 10% and 20% of the current flowing between the surface electrodes 20, 22 is propagated through an implanted conductor 24, even when there is no electrical return conductor 34. The type of current may be dependent upon the application for which the invention is intended; for example, continuous current would be applied, rather than pulsatile current, when the target body tissue is bone and promotion of bone growth is desired.

As is known to those skilled in the art, the electric currents delivered by a pulse generator to a plurality of electrodes 20, 22 may be independently controlled with the use of an interleaved pulse train. This comprises a sequence of stimulus pulses of different amplitudes, the pulses separated in time by a few milliseconds and delivered to each electrode in turn, the sequence as a whole being repeated at a rate such as 30 times per second. The amplitudes of the pulses flowing through each electrode may thereby be controlled independently.

For activation, low frequencies are generally used, for example, a frequency in the range of between 1 and 100 Hz, or alternatively, in the range of between 1 and 50 Hz. Still alternatively, the frequency can be in the range of between 1 and 20 Hz.

As an example, FIG. 2 illustrates the invention for use in the treatment of a movement disorder requiring activation of the median nerve 46. The median nerve 46 innervates most of the flexor muscles in front of the forearm, most of the short muscles of the thumb, and the short muscles of the hand. A subject's arm 48 is illustrated with the implant 18 implanted in the forearm. The electrical conductor 24 is illustrated with its pick-up end 26 forming a termination 30 for receiving the electrical current from the surface cathodic electrode 20. The stimulating end 28 forms a termination 30 for delivering the electrical current to the median nerve 46. A surface anodic electrode 22 is positioned on the skin 10. A flow of electrical current from the power source 40 is supplied via cathodic wire 42 into the skin 10 at the surface cathodic electrode 20 and the surface anodic electrode 22 via anodic wire 44. The electrical current flows through the termination 30, the pick-up end 26, the electrical conductor 24, the stimulating end 28, a portion of the median nerve 46, the tissue between stimulating end 28 and surface anodic electrode 22 including the skin underlying electrode 22, the surface anodic electrode 22, anodic wire 44 and the power source 40, thus completing the electrical circuit. Some of the current flowing between the stimulating end 28 and the surface anodic electrode 22 passes through the target body tissue (in this example, median nerve 46), thereby causing the muscle 16 of the arm 48 to be stimulated.

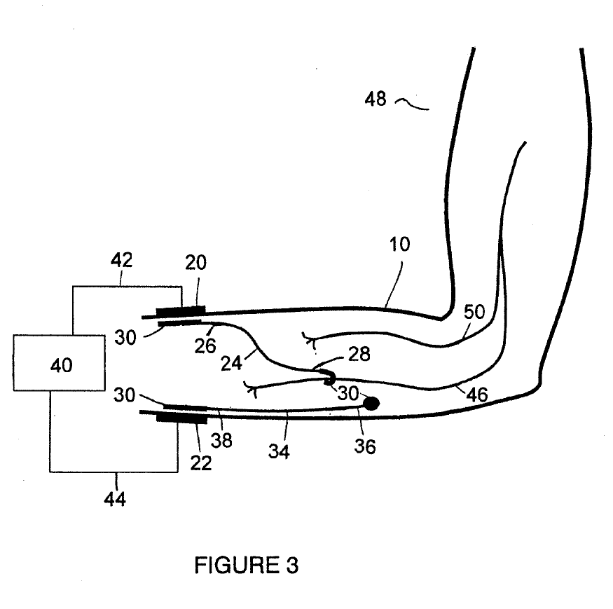

As a further example, FIG. 3 again illustrates the invention for use in the treatment of a movement disorder requiring activation of the median nerve 46. However, in addition to the components shown in FIG. 2, FIG. 3 illustrates an electrical return conductor 34. The electrical circuit is essentially the same as that described for FIG. 2, with the exception that after flowing through the stimulating end 28 and the median nerve 46, the electrical current flows through termination 30, the collecting end 36, the electrical return conductor 34, the returning end 38, termination 30, the surface anodic electrode 22, anodic wire 44 and the power source 40, thus completing the electrical circuit. Advantageously, the electrical return conductor 34 acts to collect electrical current flowing through the target body tissue (i.e., median nerve 46) from the electrical conductor 24 and provides a low impedance pathway back to the surface anodic electrode 22, thereby concentrating the electric field through the target body tissue (i.e., median nerve 46).

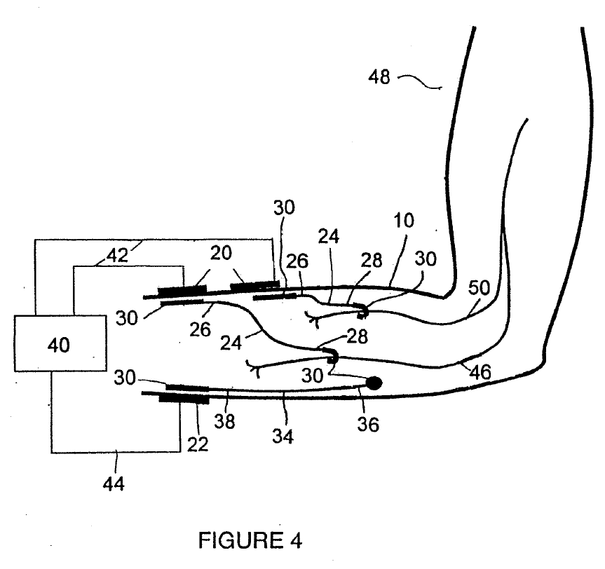

As yet a further example, FIG. 4 illustrates a plurality of implants 18 for electrically stimulating more than one target body tissue independently or in unison to activate neural impulses. Each implant 18 is implanted entirely under the subject's skin 10 and is of a sufficient length to extend to a different target body tissue. The presence of multiple implants 18 necessitates positioning of a plurality of surface cathodic electrodes 20, and one or more surface anodic electrodes 22 appropriately relative to the implants 18 to stimulate the different target body tissues independently or in unison. FIG. 4 illustrates the invention for use in the treatment of a movement disorder requiring stimulation of the median nerve 46 and the radial nerve 50. The radial nerve 50 innervates extensor muscles on the back of the arm and forearm, the short muscles of the thumb, and the extensor muscles of the index finger. Two separate surface cathodic electrodes 20 are each electrically connected via two separate cathodic wires 42 to a stimulator (not illustrated) operated by the power source 40. Electrical current is transmitted to the two separate electrical conductors 24, one of which extends to the median nerve 46, and the other to the radial nerve 50. An electrical return conductor 34 extends from the target tissue (i.e., below the median nerve 46) to subcutaneous tissue located below one surface anodic electrode 22.

The electrical path of the current is as follows: cathodic wire 42, the surface cathodic electrodes 20, the skin 10, termination 30, the pick-up end 26, the electrical conductor 24, the stimulating end 28, termination 30, the median nerve 46 and/or radial nerve 50, termination 30, collecting end 36, electrical return conductor 34, returning end 38, termination 30, surface anodic electrode 22, anodic wire 44, and power source 40. The median nerve 46 and radial nerve 50 can be stimulated either independently by pulsatile electrical current to provide firstly, a flexion or upward position of the wrist and finger closing (via the median nerve 46), then secondly, extension or downward position of the wrist and finger extension (via the radial nerve 50). Alternatively, the median nerve 46 and radial nerve 50 can be stimulated simultaneously for example, to straighten the hand (i.e., position the wrist horizontally). It will be appreciated by those skilled in the art that the invention can be applied to other target body tissues and disorders where activation of neural impulses is needed to restore normal functioning.

### C. Blockade of Neural Impulses Using the Router System

In some pathological states, action potentials are transmitted which do not serve a useful purpose; hence, blocking of unnecessary nerve impulses is required to restore normal functioning. The invention provides a method for treating disorders by applying electrical current in the form of cyclical waveforms at a frequency capable of blocking a target body tissue so as to treat the disorder. Electrical current waveforms are generated at a frequency which is high enough to cause conduction block in target neural tissues. For example, the electrical current can be applied in the form of pulses, typically 20 to 1,000 microseconds in duration at a rate high enough to cause conduction block in the target axons. The frequency and pulse parameters, including pulse amplitude, pulse duration and pulse rate, depend upon many factors that are well known to those skilled in the art; for example, the type of nerve to be blocked (either in the peripheral or central nervous system), the tissue which the nerve innervates (e.g., autonomic organs such as the bladder, or somatic organs such as muscle), the size of the nerve, the subject to be treated, the type of condition, the severity of the condition, and the receptiveness of the subject to the treatment.

A wide range of frequencies from 100 Hz to 30 kHz has been reported to produce an effective block depending upon various parameters among those described above and the particular stimulation technique used; for example, 100-300 Hz for subthalamic nucleic in human deep brain to reduce motor symptoms (Ashkan et al., 2004; Filali et al., 2004); 500 Hz for a muscle nerve (Solomonow et al., 1983); 600 Hz for a sacral nerve root in an acute spinalized dog to achieve bladder voiding (Shaker et al., 1998); 600 Hz for the ventral sacral root to inhibit urethral sphincter contractions in chronically spinalized dogs (Abdel-Gawad et al., 2001); 200-1400 Hz for epidural stimulation in a human to moderate motor disorders (Broseta et al., 1987); 4 kHz for the pudendal nerve in cats to block external urethral sphincter contractions (Tai et al. 2004, 2005); and 10-30 kHz for a peripheral nerve to treat spasticity and pain (Bhadra and Kilgore, 2005).

For blockade of neural impulses, it is required that the frequency is higher than frequencies normally required to stimulate a nerve to conduct action potentials, and high enough to block conduction of action potentials in target body tissues. In general, for blocking, high frequencies are useful, for example, the cyclical waveform can be applied at a frequency in the range of between 100 and 30,000 Hz, or alternatively in the range of between 100 and 20,000 Hz. Still alternatively, the cyclical waveform can be applied at a frequency in the range of between 100 and 10,000 Hz, or in the range between 200 and 5,000 Hz.

Example 1 (see below) illustrates use of the present invention, the results of which suggest that stimulation with an amplitude greater than 3 mA and a frequency greater than 200 Hz is capable of blocking transmission of neural impulses in the pudendal nerve of a cat. It is highly advantageous that the stimulator of the invention is external to the subject's body and supplies high frequency electrical current waveforms to the surface cathodic and anodic electrodes 20, 22 positioned externally on the subject's skin. A wide range of pulse parameters can be readily and easily tested and adjusted to determine optimal parameters for achieving the desired physiological result in a subject following implantation of the electrical conductor 24.

Exemplary pulse parameters of high frequency trains of electrical current flowing between surface cathodic and anodic electrodes 20, 22 are as follows: current-controlled or voltage-controlled biphasic pulses, with phase durations ranging from 10 microseconds to 1,000 microseconds, or cyclical waveforms such as sinusoids or triangular, rectangular or sawtooth waveforms.

Blockade of a nerve impulse using the invention is reversible at all frequencies such that when high frequency stimulation is turned off, the nerve can again propagate action potentials and no damage has been incurred. Further, partial or complete blocking of a nerve impulse can be achieved depending upon the condition to be treated. For example, complete blocking of sensory nerves may be required to alleviate pain, while partial or complete blocking of sensory and motor nerves may be needed to reduce spasticity.

Other embodiments of the invention are possible. For instance, a plurality of implants 18 for electrically blocking more than one target body tissue independently or in unison can be used. The presence of multiple implants 18 necessitates positioning of a plurality of surface cathodic electrodes 20, and one or more surface anodic electrodes 22 appropriately relative to the implants 18 to block the different target body tissues independently or in unison.

In another embodiment, a plurality of implants 18 for electrically activating neural impulses in more than one body tissue independently or in unison can be used concomitantly with the above implants 18 for electrically blocking neural impulses in target body tissues. Two separate signals are required, with a low frequency signal required to activate a nerve, and a high frequency signal required to block another nerve. For example, bladder voiding can be achieved by applying low frequency pulse trains to the sacral nerve root S1, which elicits bladder and sphincter contractions, and by simultaneously applying high frequency waveforms to the pudendal nerve to block the sphincter contractions induced by stimulating the sacral nerve root S1.

Various disorders requiring blocking of neural impulses are amenable to treatment by the invention as shown in FIG. 1. As an example, the invention can be used to achieve bladder voiding (see Example 1). When the bladder is full, nerve signals are normally sent to the brain to convey the need to urinate. In response, the brain initiates a coordinated response in which the bladder wall contracts, creating pressure that forces urine into the urethra, while a sphincter, surrounding the urethra, opens to allow urine to flow out. In certain disorders, for example spinal cord injury, the bladder is generally unable to empty because of hyper-reflexive contractions of the external sphincter. The closure of the sphincter is maintained by reflexes intended to maintain continence, which can no longer be suppressed by signals from the brain. The pudendal nerve innervates the musculature of the pelvic floor and the external urethal and external anal sphincters. The motor component of the urinary branch of the pudendal nerve activates the external urethral sphincter muscle. Blocking this branch relaxes the sphincter and allows bladder emptying.

To achieve bladder voiding, the electrical conductor 24 is implanted in the subject with its stimulating end 28 positioned proximate or in contact with the pudendal nerve. The pick-up end 26 of the electrical conductor 24 extends into subcutaneous tissue located below the surface cathodic electrode 20. The surface cathodic and anodic electrodes 20, 22 are positioned preferably on the subject's skin above the hips. Since the pudendal nerve is present on both the left and right sides of the body, two electrical conductors 24 can optionally be positioned on both sides to achieve blocking. This would necessitate one surface anodic electrode 20, and either one or two surface cathodic electrodes 22. The electrical conductor 24 provides a conductive pathway for at least a portion of the electrical current flowing between the surface cathodic and anodic electrodes 20, 22 in the form of high frequency waveforms and transmits that portion of the electrical current to the pudendal nerve. Blocking of the pudendal nerve by stimulation with high frequency electrical pulses subsequently causes the urethal sphincter to open (as observed by a sudden large drop in intraurethral pressure), allowing bladder voiding. The pudendal nerve is blocked to allow bladder voiding until the bladder is empty.

The invention can also be used to alleviate pain, which generally refers to a localized sensation of discomfort resulting from the stimulation of specialized nerve endings. Peripheral nerves are nerves and ganglia outside the brain and spinal cord. In a mixed peripheral nerve, the thinnest exteroceptive sensory fibers convey impulses which are interpreted in sensation as pain. The present invention can thus be used to block sensory axons in peripheral nerves to reduce pain. For example, trigeminal neuralgia is a repeated and incapacitating pain affecting the lower portion of the face and arising from malfunction of the trigeminal nerve, which carries sensory information from the face to the brain and controls the muscles involved in chewing. The electrical conductor 24 is implanted having its pick-up end 26 proximate or in contact with a cranial nerve (such as the trigeminal nerve) and its stimulating end 28 positioned subcutaneously within the head. Surface cathodic and anodic electrodes 20, 22 are positioned on the skin of the head. The electrical conductor 24 provides a conductive pathway for at least a portion of the electrical current flowing between the surface cathodic and anodic electrodes 20, 22 in the form of high frequency cyclical waveforms transmits that portion of the electrical current to the trigeminal nerve. Blocking of the trigeminal nerve may subsequently reduce pain in patients with trigeminal neuralgia.

Spasticity, tremor and/or muscle weakness is an example of a further disorder to which the invention is applicable for blocking of neural impulses. Spasticity is characterized by a state of hypertonicity (i.e., an excessive tone of skeletal muscle with heightened deep tendon reflexes), and can cause muscle stiffness and awkward movements. It can occur as a result of stroke, cerebral palsy, multiple sclerosis or spinal cord injury. Nerve fibers involved with spasticity include sensory and motor nerves. The present invention can be used to block sensory and motor nerves to block muscle spasms. Referring again to FIG. 3, the median nerve 46 can be blocked (rather than activated as previously described) to alleviate flexure spasms occurring due to a stroke or multiple sclerosis. A flow of electrical current from the power source 40 is supplied in the form of high frequency cyclical waveforms via cathodic wire 42 into the skin 10 at the surface cathodic electrode 20 and the surface anodic electrode 22 via anodic wire 44. The electrical current flows through the termination 30, the pick-up end 26, the electrical conductor 24, the stimulating end 28, a portion of the median nerve 46, the tissue between stimulating end 28 and surface anodic electrode 22 including the skin underlying electrode 22, the surface anodic electrode 22, anodic wire 44 and the power source 40, thus completing the electrical circuit. Some of the current flowing between the stimulating end 28 and the surface anodic electrode 22 passes through the target body tissue (in this example, median nerve 46), thereby blocking nerve impulses along the median nerve 46 and preventing contraction of the muscle 16 of the arm 48.

The invention can also be used to reduce pain and spasticity by blocking the spinal cord. As an example, back pain or leg muscle spasms may be alleviated by blocking spinal nerves in the lumbar spine. The lumbar spinal nerves (L1 to L5) supply the lower parts of the abdomen and the back, the buttocks, some parts of the external genital organs, and parts of the legs. The electrical conductor 24 is implanted with its stimulating end 28 positioned between lumbar vertebrae into the lumbar spinal canal. The stimulating end 28 is placed proximate to the epidural space between the dura mater and the walls of the spinal canal. The pick-up end 26 is positioned subcutaneously in the lower back of the body. Surface cathodic and anodic electrodes 20, 22 are positioned on the skin of the lower back. The electrical conductor 24 provides a conductive pathway for at least a portion of the electrical current flowing between the surface cathodic and anodic electrodes 20, 22 in the form of high frequency cyclical waveforms and transmits that portion of the electrical current to the spinal cord. Blocking of the lumbar spinal nerves may subsequently reduce pain or spasticity in affected regions of the lower body.

The invention can be used to treat pathological tremor, Parkinson's disease, dystonia and other disorders by blocking deep brain nuclei. Such target tissues can include the basal ganglia which includes the subthalamic nucleus and substantia nigra. Parkinson's disease is a disorder of the basal ganglia. The electrical conductor 24 is implanted with its stimulating end 28 positioned proximate or in contact with the basal ganglia. The pick-up end 26 is positioned subcutaneously within the head. Surface cathodic and anodic electrodes 20, 22 are positioned on the skin of the head. The electrical conductor 24 provides a conductive pathway for at least a portion of the electrical current flowing between the surface cathodic and anodic electrodes 20, 22 in the form of high frequency waveforms and transmits that portion of the electrical current to the basal ganglia. The electrical current blocks the electrical signals that cause symptoms of movement disorders. The present invention may thus be useful in blocking the basal ganglia or other target deep brain nuclei to treat disorders in which movement is impaired.

The invention is further illustrated in the following non-limiting Example. Two experiments were performed on anesthetized cats using the present invention to achieve high-frequency blockade of the pudendal nerve.

### Methods

Surgical procedures: Cats were pre-operatively medicated with acepromazine (0.25 mg/kg intramuscular), glycopyrrolate (0.01 mg/kg intramuscular) and buprenorphine (0.01 mg/kg intramuscular) and anesthetized with a mixture of isoflurane (2-3% in carbogen, flow rate 2 L/min). The trachea was cannulated and connected to a closed loop anesthetic system that monitored respiration rate and assisted ventilation. One jugular or cephalic vein was catheterized to allow administration of fluids and drugs. The bladder was exposed via a midline abdominal incision and catheterized to allow the addition and withdrawal of fluids and the measurement of pressure within the bladder with a pressure transducer (see below). A second catheter (Kendall, closed end Tom Cat catheter) was inserted into the urethra and connected to a second pressure transducer to allow measurement of intraurethral pressure. The pudendal nerves were exposed by incisions lateral to the base of the tail. Cuff or hook electrodes were placed on the pudendal nerve or its branches. At the end of the experiment, the animals were euthanized with Euthanyl™.

Pressure measurements: Bladder pressure and urethral pressure were monitored in most stimulation trials. The urethral catheter was attached to a Harvard Apparatus Pump 22 syringe pump and set to infuse saline at 0.2 mL/min to allow measurement of intraurethral pressure as per the method of Brown and Wickham. Both the bladder and urethral catheters were connected via Luer ports to Neurolog NL108D4/10 domes and NL108T4 isolated pressure transducers. The pressure signals were low-pass filtered at 30 Hz and sampled at a rate of 100 samples per second using a CED 1401 laboratory computer interface and sampling software.

Stimulators: Neurolog (Digitimer Ltd., Welwyn Garden City, UK) modules NL304 (period generator), NL403 (delay-width), NL510 (pulse buffer) and NL800 (stimulus isolator) were used to deliver constant current monophasic pulses and Grass (Grass-Telefactor, West Warwick, R.I., USA) SD9 and S48 stimulators were used to deliver constant voltage monophasic pulses.

Means of delivering stimulation: Two types of stimulation were tested, namely direct stimulation, and stimulus routing using the present invention. In direct stimulation, a stimulating electrode was placed on the exposed pudendal nerve and connected via an insulated lead wire to the cathodal output of the Grass stimulator. A second (indifferent) electrode comprising an alligator clip attached to the incised skin near the exposed pudendal nerve was connected to the anodal output of the Grass stimulator. In stimulus routing using the present invention, an implanted electrode comprising a pick-up end in the form of a metal disk or coiled wire connected via an insulated lead wire to a stimulating end was implanted so that the pick-up end was located subcutaneously over the lumbar spine under a surface cathodal electrode and the stimulating end was in contact with a pudendal nerve. The surface cathodal electrode was a conductive gel electrode (Kendall, H59P) applied to the shaved skin overlying the pick-up end and connected to the cathodal output of the Neurolog stimulator. A second surface electrode was placed a few centimeters rostral to the cathodal electrode and connected to the anodal output of the Neurolog stimulator.

Low frequency (20 Hz) direct stimulation via a hook electrode placed proximally on the pudendal nerve was used to elicit contractions of the external urethral sphincter. These contractions were monitored in terms of intraurethral pressure as increases in intrauthreal pressure are indicative of contractions of the external urethral sphincter. During periods of low-frequency direct stimulation, bursts of high-frequency stimulation were delivered via the router system through a hook electrode placed more distally on the pudendal nerve. The efficacy of the router-mediated high-frequency stimulation in blocking the nerve activity evoked by the direct low frequency stimulation was thereby determined.

### RESULTS

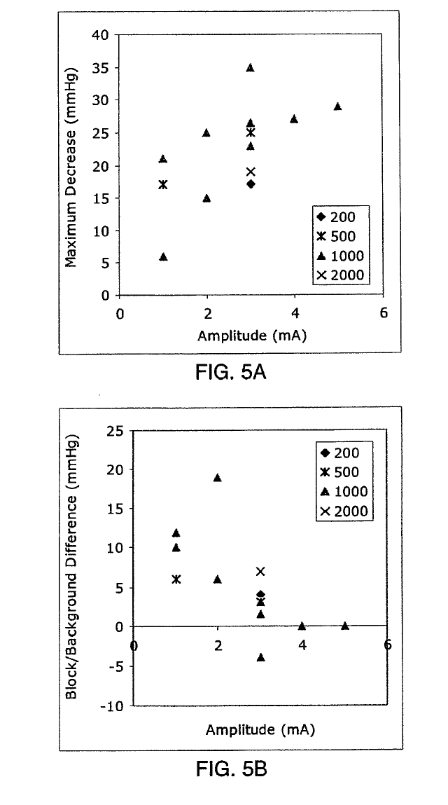

FIGS. 5A and 5B shows the results obtained in one animal when stimulation frequency and amplitude were varied and the efficacy of the pudendal nerve block was observed. The efficacy was measured by observing changes in the intraurethral pressure with the open port of the intraurethral catheter placed in the region of the external urethral sphincter. The right pudendal nerve was stimulated proximally at low frequency to elicit external urethral sphincter contractions while high frequency stimulation was applied distally to block the contractions. The maximum decrease in intraurethral pressure (FIG. 5A) was defined as the difference between the intraurethral pressure immediately before high frequency stimulation was applied and the minimal pressure obtained during high frequency stimulation. There appeared to be a trend towards larger decreases in intraurethral pressure at higher stimulation amplitudes. Blocking was obtained at all stimulation frequencies examined (i.e., 200, 500, 1000 and 2000 Hz).

FIG. 5B summarizes the effect of stimulation pulse frequency and amplitude on the ability of high frequency stimulation to return the intraurethral pressure to baseline. This provides a measure of the completeness of the block. The most complete blocking was achieved with stimulation amplitudes of 3 mA and higher. At a stimulation pulse amplitude of 3 mA, all tested frequencies (i.e., 200, 500, 1000 and 2000 Hz) elicited a nearly complete block. There was a general trend towards a more complete block at higher stimulation pulse amplitudes. A Y-axis value of zero indicates that the intraurethral pressure during high frequency stimulation was equal to the pre-stimulation baseline pressure.

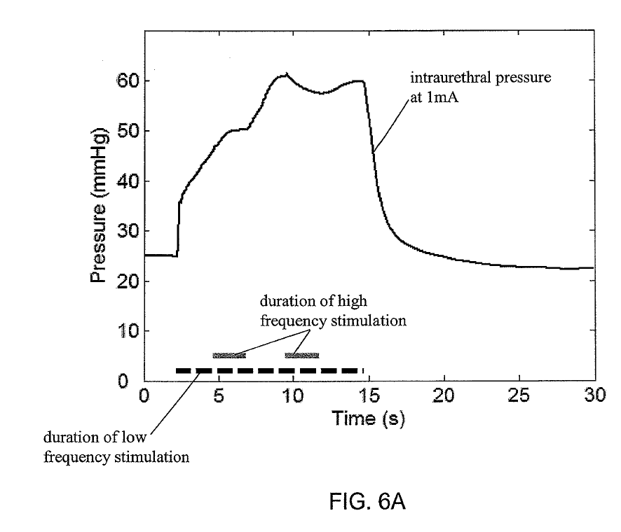

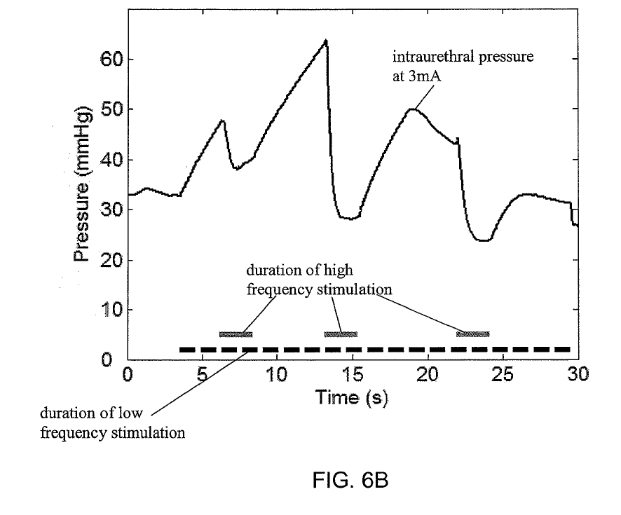

FIGS. 6A and 6B show the effect of stimulation pulse amplitude on pudendal nerve blocking in one animal. The traces represent intraurethral pressure obtained at 1 mA (FIG. 6A) and at 3 mA (FIG. 6B), the dashed bars indicate duration of low frequency stimulation and the solid bars indicate the duration of high frequency stimulation. Low frequency stimulation was applied proximally on the pudendal nerve with a monopolar hook electrode directly connected to the cathodal output of the Grass stimulus generator. High frequency stimulation was applied distally on the pudendal nerve with a monopolar hook electrode connected to the Neurolog stimulus generator via the stimulus routing system of the present invention. The anodal indifferent surface electrode was placed a few centimeters rostral to the cathodal surface electrode. Low frequency stimulation was delivered at a frequency of 20 Hz with pulses having an amplitude of 520 μA and a pulse width of 300 μs. High frequency stimulation was delivered at a frequency of 1 kHz with pulses having a pulse width of 100 μs. Stimulation with 1 mA pulse amplitudes had very little effect on the intraurethral pressure and elicited very little block of sphincter activity. However, with the stimulation pulse amplitude increased to 3 mA, a complete temporary and reversible block of sphincter activity was achieved.

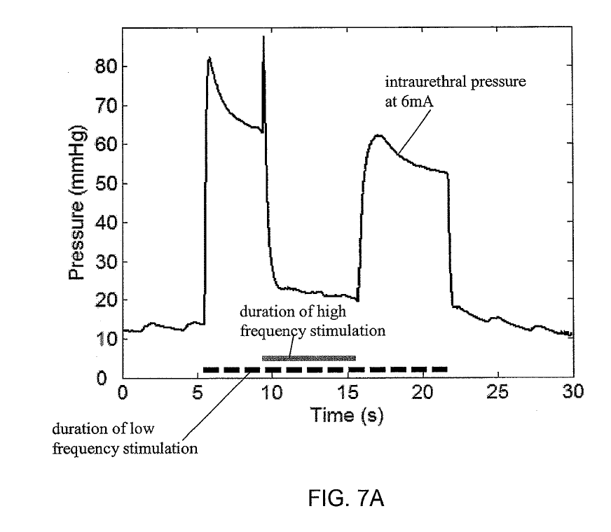

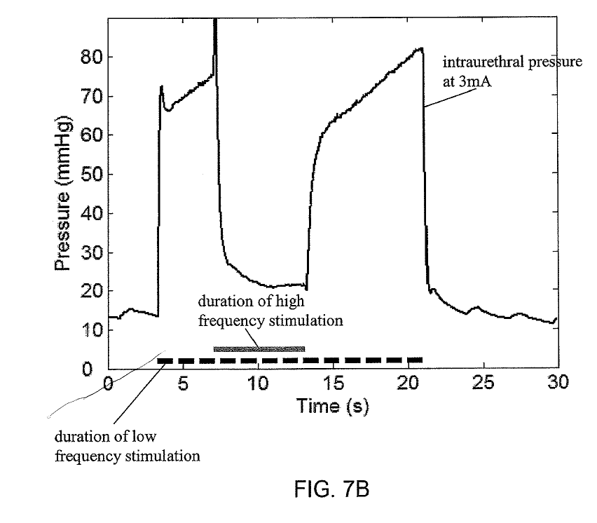

In the trials shown in FIGS. 7A and 7B, the stimulation frequency was 2 kHz. At 6 mA pulse amplitudes (FIG. 7A), a nearly complete block was achieved, but contractions of the leg under the surface cathodal electrode accompanied the stimulation. These contractions were maintained for the duration of the stimulation. At pulse amplitudes of 3 mA, similar blocking efficacy was achieved without concomitant leg contractions (FIG. 7B). In this trial, low frequency stimulation (duration indicated by dashed bars) was delivered at a frequency of 20 Hz with pulses having an amplitude of 300 μA and a pulse width of 200 μs, while high frequency stimulation (duration indicated by solid bars) was delivered at a frequency of 2 kHz with pulses having a pulse width of 150 μs.

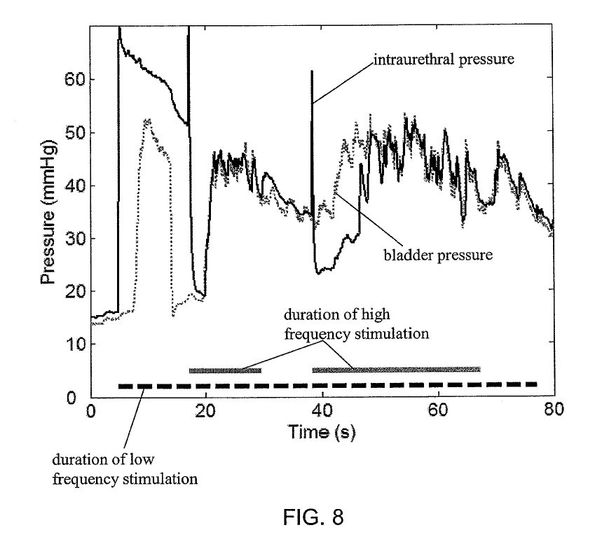

In further trials, in addition to low frequency stimulation of the proximal pudendal nerve and high frequency stimulation of the distal pudendal nerve, increases in bladder pressure were generated by manually applied abdominal pressure. FIG. 8 shows an example where this combined procedure was performed. FIG. 8 shows the effect of pudendal nerve blocking on intraurethral pressure in one animal. The solid trace is intraurethral pressure, the dotted trace is bladder pressure, the dashed bar indicates duration of low frequency stimulation and the solid bars indicate duration of high frequency stimulation. Low frequency stimulation was delivered at a frequency of 20 Hz with pulses having an amplitude of 330 μA and a pulse width of 200 μs. High frequency stimulation was delivered at a frequency of 2 kHz with pulses having an amplitude of 4 mA and a pulse width of 100 μs. Initial low frequency stimulation was used to generate an external urethral sphincter contraction after which the bladder pressure was increased by manual application of pressure to the abdomen. No voiding occurred during the first 20 seconds as intraurethral pressure was maintained higher than the manually generated bladder pressure by the direct pudendal nerve stimulation. Once high frequency stimulation of the distal pudendal nerve was applied, intraurethral pressure became equal to bladder pressure, indicating that the external sphincter was relaxed, and voiding occurred.

Several trials were performed in which the intraurethral catheter was removed to examine voiding. Complete voiding was achieved when high frequency stimulation was used to block low frequency stimulation-induced external urethral sphincter contractions and the bladder pressure was increased manually. In general, the results above suggest that use of the present invention and stimulation with an amplitude greater than 3 mA and a frequency greater than 200 Hz contributes to blocking transmission of activity in the pudendal nerve. Determination of stimulation parameters to produce an optimal block is under investigation. It will be understood by those skilled in the art that other stimulation parameters may produce better blocking results, particularly in other parts of the peripheral and central nervous systems. It will also be understood that it will be desirable to determine the stimulation parameters required to produce optimal nerve blocking on an individual basis, as these parameters may vary from subject to subject, depending upon the characteristics of the skin as well as the precise positioning of the components of the present invention.

### D. Advantages of the Router System

As described above, the invention thus provides several advantages, primarily the capability of stimulating a target body tissue to either activate or block neural impulses depending upon the frequency and the disorder to be treated. Further, the present invention includes a means of “remote” stimulation, that is the surface cathodic and anodic electrodes 20, 22 do not have to be positioned over target body tissues. Remote target body tissues, such as nerves 12, can be stimulated to activate or block neural impulses from closely spaced surface cathodic and anodic electrodes 20, 22, by routing current through separate electrical conductors 24 simultaneously to several remote target body tissues.

Further, greater selectivity is provided in stimulating target body tissues to activate or block neural impulses. The electrical conductor 24 extends to a specific target body tissue, or multiple electrical conductors 24 can extend to multiple target body tissues. Stimulation is thus specific to the target body tissues, and stimulation of non-target body tissues is avoided. As an electrical conductor 24 of sufficient length is used to reach target body tissues, stimulation of target body tissues which are positioned deep within the body or organs such as the muscles, brain, cochlea, optic nerve, heart, bladder, urethra, kidneys and bones, can be achieved.

Stimulation to activate or block neural impulses is reproducible at will. The electrical conductor 24 is passive and can remain permanently implanted with the pick-up end 26 under the skin 10 beneath the site at which the surface cathodic electrode 20 would be placed, and the stimulating end 28 positioned proximate to the target body tissue. To the inventor's knowledge, difficulty has been encountered in positioning surface electrodes accurately to obtain acceptable selectivity of stimulation of body tissues. The inventor has discovered that surprisingly, the invention requires far less accuracy in positioning of the surface cathodic and anodic electrodes 20, 22; consequently, stimulation of body tissues to activate or block neural impulses is more accurately reproducible.

Further, the invention avoids problems inherent in other forms of stimulation. The conductors (i.e., electrical conductor 24, electrical return conductor 34) do not emerge through the skin, thus reducing the risk of infection which may arise with percutaneous devices. There is no need to construct an implant housing its own stimulator, signal generator or power source, or to provide radio-frequency or other telemetric command signals through the skin.

### E. Controlled Nerve Ablation

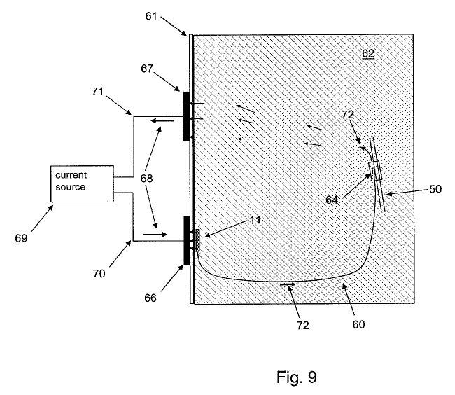

A preferred method of blocking nerve conduction through controlled nerve ablation is described below with reference to FIGS. 9-12, in which one or more implants in the form of passive conductors, multiple surface electrodes and external or implanted current sources are described. These components may take the form of the similar components described hereinabove with respect to the applications for stimulation and blocking. However, it will be understood that the method and system of the invention as it relates to controlled nerve ablation to treat unwanted or overactive nerve activity in a subject, is not limited to the system components of the figures.

Referring to FIG. 9, there is illustrated one application in which a target nerve 50 is partially or fully ablated with a single implanted, insulated passive conductor 60. The insulated conductor 60 (implant) has a first uninsulated end 63 (which serves as a pick-up portion) and a second uninsulated end 64 (which serves as a delivery portion). Ends 63, 64 having terminations of specific shapes and composition that maximize the electrical conductance of the interface with the bodily tissues, as described hereinabove. The insulated conductor 60 when implanted is entirely under the subject's skin 61 within bodily tissue 62. First end 63 is positioned in the subcutaneous tissue under the skin 61. Second end 64 is positioned in close proximity to, or attached to, the target nerve 50. A suitable nerve cuff 65 may be used to secure the second end 64 to the target nerve 50 to prevent migration. A first surface electrode 66 is positioned on the subject's skin, in direct electrical contact with the subject's skin, over the first end 63. Similarly, a second surface electrode 67 is placed in direct electrical contact with the subject's skin, preferably in spaced relationship to electrode 66. Electrical current 68 is introduced from a current source 69 into the skin 61 between lead wires 70 and 71 and surface electrodes 66 and 67. A portion 72 of the current 68 enters the first uninsulated end 63 of implanted conductor 60 and flows to the second uninsulated end 64 and then through the bodily tissue 62, including through the target nerve 50, back to surface electrode 67, wire 71 and the current source 69, thus completing the electrical circuit. The portion 72 of the current which flows to the conductor 60 and the nerve 50 is largely via resistive coupling, with a minor capacitive coupling component. In general, in most applications approximately 10 percent of the current delivered by the surface electrodes will flow in the implanted conductor, although this percentage will vary with such factors as the depth of implanting, the size and configuration of the surface electrodes, the size and shape of electrical terminations at the ends 63, 64 of the conductor 60, and the proximity to the nerve 50. The current source 69 is adapted to deliver direct current or pulsatile current in a manner such that a net charge is delivered to the target nerve 50. The portion of the current 72 passing through the nerve 50 has current parameters including current level and duration which are controlled at the current source 69 to cause controlled nerve ablation to the target nerve 50 until the unwanted or overactive nerve activity is reduced in the target nerve 50 and/or in surrounding body tissue such as an affected muscle.

Various conductive materials can be used at the pick-up end and the delivery end of the implanted conductor(s). The preferred materials for the pick-up and delivery ends are noble metals such as platinum or alloys of platinum and iridium, as these metals are less subject to dissolution than metals such as stainless steel.

While direct current is the preferred form of current for achieving controlled nerve ablation, alternatives to the above nerve ablation method and system include delivering charge imbalanced time varying current or charge imbalanced pulsatile current. For example pulses of current in one direction only, or biphasic pulses in which the charge delivered in one phase exceeds that in the other phase might be used. To avoid skin inflammation and irritation when using charge-imbalanced stimulation, it is recommended that the surface electrode whose mean voltage is more positive with respect to the other surface electrode, be made as large as possible so as to reduce the current density at the skin surface.

Regarding the preferred parameters of pulsatile current applied to the nerve, it has been reported that imbalanced biphasic pulsatile stimulation above a net direct current of 5 mA/cm², and monophasic pulsatile stimulation above a net direct current of 2 mA/cm² cause damage to nerve and muscle tissues (Scheiner et al. 1990). Regarding the parameters of charge density and charge per phase of pulsatile stimulation, the range of combinations that are sufficient to cause nerve damage are shown in FIG. 8 of Scheiner et al. (1990).

As an alternative to using a nerve cuff at the delivery end of the conductor, the delivery end of the conductor might be positioned proximate the nerve, or held in place at the nerve or surrounding bodily tissue with tines, protuberances or the like. The electrical terminations at the delivery end are configured to deliver the current densities sufficient to cause controlled nerve ablation. The conductor may alternatively extend percutaneously through the subject's skin, instead of being entirely implanted, and may be directly connected to an external current source. Alternatively, the current source itself might be implanted. An implanted current source can be inductively coupled to an external controller. Alternatively, the implanted current source might be controlled externally through wireless communication, or the conductor may be configured to carry controls. Multiple implanted conductors may be used, either to provide a return path for the electrical current, or to deliver controlled nerve ablation to different target nerves. The surface electrodes may be provided in pairs or otherwise to resistively couple to one or more implanted conductors.

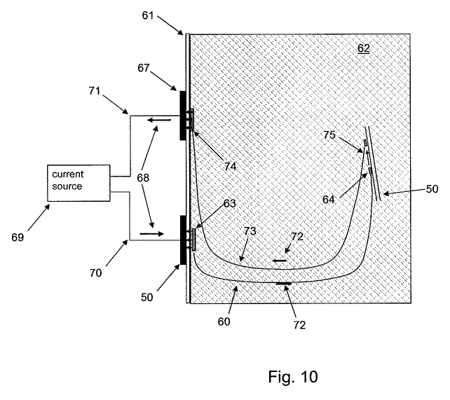

Referring to FIG. 10, a second application is shown in which the electrical current delivered to the target nerve 50 flows between two implanted conductors 60, 73 (like components being with like reference numerals to FIG. 9) adjacent to the target nerve 50. The advantage of using two conductors instead of one, is that a second uninsulated end 75 of the second conductor 73 acts to collect electrical current flowing from uninsulated end 64 of the first conductor 60, thereby concentrating the current 68 in the vicinity of target nerve 50. The first conductor 60 is provided as described above for FIG. 9, and a second conductor 73 is provided as a return conductor. The second conductor 73 has a first uninsulated end 74 and a second uninsulated end 75, ends 74, 75 having terminations of specific shapes and composition that maximize the electrical conductance of the interface with the bodily tissues, as explained above. First end 63 of conductor 60 is implanted under the subject's skin 61 and second end 64 is positioned in close proximity to nerve 50 as described above. First uninsulated end 74 of conductor 73 is implanted under the skin 61 in close proximity the second surface electrode 67. Second uninsulated end 75 of conductor 73 is implanted in the vicinity of uninsulated end 64 of conductor 60. It will be understood that the shape, composition and spatial arrangement of ends 64 and 75 may vary. For example, both of the ends 64, 75 may take the form of small, closely-spaced contacts within an insulating cuff placed around the nerve 50. Similarly it will be understood that the positions of the surface electrode sites may vary, depending on the location and nature of the target nerves 50.

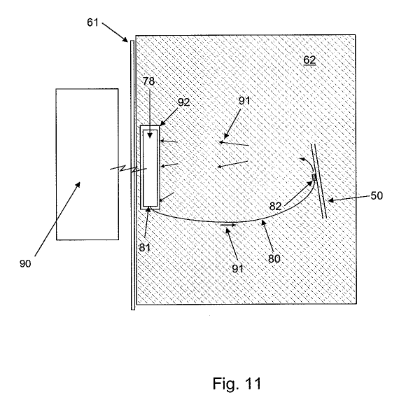

Referring to FIG. 11, there is illustrated an application in which the electrical current that is delivered to the target nerve 50 is supplied by an implanted current source 78 connected to a single implanted conductor 80. The insulated conductor 80 has a first end 81 and a second uninsulated end 82. An implantable current source 78 is provided having an output terminal (not shown). The current source 78 is adapted to deliver direct current or pulsatile current with control of the current level and duration parameters. To that end, current source 78 may take the form of a implanted battery with wireless external control, or more preferably a implanted current source that is inductively coupled and powered by an external controller 90, as shown in Figure. The first end 81 of the conductor 80 is connected to the output terminal 79 of the current source 78, and the connection is insulated. This connection and insulating may take place during the manufacture of conductor 80 and current source 78, or it may take place during implantation, with the use of an insulating sleeve (not shown). The current source 78 is embedded in bodily tissue under the skin 61. Current source 78 is positioned in a convenient location under the skin 61. Second end 84 of conductor 80 is positioned in close proximity to target nerve 50. The implanted current source 78 is controlled with the external inductively-coupled controller 90 to deliver current 91 through conductor 80 and uninsulated end 82 to nerve 50 and surrounding bodily tissue 62. Delivered current 91 flows through the nerve 50 and surrounding bodily tissue 62 back to current source 78, thus completing the electrical circuit. In one embodiment current source 78 has a metal case 92 that provides the return current path to the circuitry of current source 78.

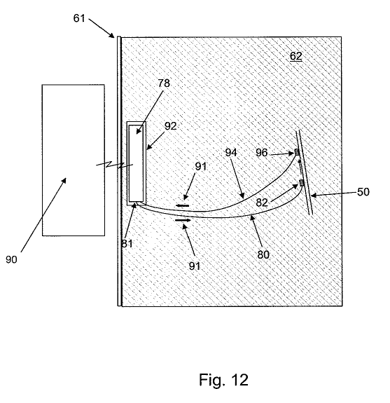

Referring to FIG. 12, there is illustrated an application in which the electrical current that is delivered to the target nerve 50 is supplied by an implanted current source 78 connected to two implanted conductors 80, 94 with uninsulated ends 82, 96 terminations adjacent to the nerve 50. The first conductor 80 has first end 81 and second uninsulated end 82. The second conductor 94 has first end 95 and second uninsulated end 96. As above, the implantable current source 78 is connected to the first end 81 of conductor 80 through the output terminal of the current source 78. The first end 95 of conductor 94 is connected to the reference terminal (not shown) of current source 78. These connections to the current source 78 are insulated are insulated prior to or during implantation as discussed above. The current source 78 and the insulated conductors are implanted under the subject's skin 61. Current source 78 is positioned in a convenient location under the skin 61. End 82 of conductor 80 and end 96 of conductor 94 are positioned in close proximity to nerve 50. The implanted current source 78 is controlled with an external inductively-coupled controller 90 to deliver current through conductor 80 to nerve 50 and surrounding bodily tissue 62. Delivered current 91 flows through the nerve 50 and surrounding bodily tissue 62 and is collected by conductor 94 through end 96. Conductor 94 delivers said current 91 back to the current source 78, thus completing the electrical circuit. The current passing through nerve 50.

The method and system of the present invention may be practiced with nerves of the central nervous system, or with peripheral nerves. Exemplary peripheral nerves to be treated include without limitation a facial nerve, a spinal accessory nerve, a musculocutaneous nerve, a median nerve, a pudendal nerve, a sciatic nerve, a femoral nerve, or branches of any of these nerves.

As indicated above, the parameters of the electrical current are controlled with respect to current amplitude and duration to obtain the desired amount of controlled nerve ablation until unwanted or overactive nerve activity is reduced in one or both of the target nerve or in a surrounding body tissue. The parameters will vary with the particular nerve, condition being treated, the size of terminations on the conductor at the pick-up end and at the nerve delivery end, and the distance from the delivery end to the target nerve. The experiments set out below illustrate both the success of the method, and can be used to determine the current parameters for a particular application and subject's conditions, as discussed below.

### EXPERIMENT 1

### Controlled Nerve Ablation in Anesthetized Rabbits

In experiments performed in anesthetized rabbits, partial or complete blockade of conduction in the sciatic and common peroneal nerve was achieved by placing a first (blocking) nerve cuff containing a metal electrode of 0.5 cm² surface area on the nerve and delivering feedback-controlled continuous direct current through the electrode to the nerve, with the use of a remote reference electrode on the skin. Numerous combinations of current amplitude and duration were explored, in the range 0.2 mA to 6 mA and 1 minute to 45 minutes. The extent and duration of conduction block caused by the direct current were measured indirectly, by observing the extent and duration of attenuation of muscle twitches elicited by pulsatile stimuli delivered to the nerve by a second nerve cuff proximal to the blocking nerve cuff. The amount of attenuation of twitches and how much recovery occurred after direct current delivery ceased, depended on both the amplitude of the direct current delivered to the nerve and its duration. For example, 0.2 mA delivered for 5 minutes or 0.5 mA delivered for 2 minutes both caused about 50% attenuation, with a complete recovery of twitch forces within 30 seconds of cessation of the current. A current of 0.3 mA delivered for 105 minutes caused 60% attenuation and no recovery for the rest of the experiment, lasting several hours. A current of 0.5 mA delivered for 20 minutes caused 100% attenuation and no recovery for the rest of the experiment, lasting several hours. These results indicate that the amount of attenuation in these experiments depended on both the level of current and its duration. The product of current and duration is the total net charge delivered. In the above examples, a current of 0.2 mA for 5 minutes corresponds to 0.06 coulomb of charge, which caused 50% attenuation followed by recovery. A current of 0.5 mA applied for 2 minutes, which also corresponds to 0.06 coulomb of charge, also caused 50% attenuation. This indicates that the same net charge delivered more rapidly caused similar amounts of nerve block. Furthermore, as charge density is also an important factor in blocking and ablating nerves, for a given current, delivery ends with smaller surface areas will require less current and shorter durations to ablate nerves and conversely delivery ends with larger surface areas will require longer currents and durations. Histology of nerves in which 100% attenuation had been achieved, showed evidence of nerve ablation in the form of ruptured axons at the nerve cuff site.

Two other factors determine whether direct current or time-varying imbalanced current cause nerve ablation. Merrill et al. (2005) includes a Figure summarizing the results of several previous studies, showing that above a certain charge density per pulse, current pulses become “unsafe.” Charge density is inversely proportional to electrode surface area, so for a given amount of charge delivered per pulse, the smaller the electrode, the greater the charge density per pulse. For pulses of the same duration, charge density is proportional to current density, so while Merrill et al. do not predict the safe limits of current densities for direct current application, it is reasonable to assume that the higher the current density, the lower both the current amplitude and duration that causes nerve ablation. Second, the further the delivery terminal on the conductor is from the nerve, the larger is the amplitude of direct current required to block the nerve reversibly (Bhadra & Kilgore 2004). Thus, the further the delivery terminal is from the nerve, the larger is the amplitude of direct current required to ablate the nerve. In the case of a lesioning electrode comprising an implanted lead whose delivery terminal is close to the nerve but not in contact with it, as may occur when a lead is inserted percutaneously without a precise knowledge of the location of the nerve, larger currents and presumably longer durations are required to ablate the nerve than if a nerve cuff is used with the delivery electrodes in contact with the nerve. Furthermore, in the case of an implanted lead receiving current through the skin from an external surface electrode, the currents delivered through the skin by this external electrode to achieve the above results have been found to be around 10 times larger, assuming that the implanted lead only picks up 10% of this current, as has been experienced with the present invention.

Thus, for current delivered through the skin through two surface electrodes and picked up by an implanted conductor as described in FIGS. 9 and 10 above, about 10% of the current is picked up by the implanted conductor or conductors. Thus to deliver from 0.3 mA to 1.0 mA to the nerve, from about 3 mA to about 10 mA is delivered through the surface electrodes.

### EXPERIMENT II

### Controlled Nerve Ablation in Anesthetized Rabbits

In a further animal experiment with a cat, nerve cuffs were implanted chronically. Over a period of several months, every two weeks, the amplitude and duration parameters were systematically explored during short periods of anesthesia, attenuation again being measured in terms of the decline in twitch forces. The results were similar to those described above for Experiment I. In the final experiment, 100% twitch attenuation was achieved by delivering 0.5 mA for 45 minutes (which corresponds to 1.35 coulomb of charge) through a delivery terminal of surface area 0.5 cm². There were no signs of fasciculation or pain or discomfort after recovery from anesthesia after any of these procedures, even though the nerve had been temporarily or permanently blocked through nerve ablation. This indicated that although many axons were destroyed, including both motor and sensory axons, none were left in a spontaneously active state, as can occur after injections of phenol. There was no evidence of dysesthesia. The 100% attenuation in the final experiment lasted for about 3 months, after which small muscle twitches could again be elicited from the proximal cuff, indicating that some nerve regeneration and associated muscle re-innervation had begun.

In humans the distance between the lesioning site and the innervated muscle or organ would generally be larger than in these animal experiments, so the time to first re-innervation would be expected to be longer.

In a clinical application, the parameters of amplitude and duration of pulsatile or direct current applied to the nerve can be determined experimentally in each individual. A recommended procedure involves gradually increasing one or both of the parameters of amplitude and duration of the current until the desired amount of attenuation of the unwanted or overactive nerve activity is observed. The desired outcome is a reduction to a desired level in the unwanted symptom, such as pain or hypertonus, or a reduction in muscle responses to test stimuli applied to the nerve proximal to the delivery end of the implanted conductor. If a reversible blockade lasting a relatively short time, such as seconds or minutes, is desired, direct currents in the range 0.2 to 0.5 mA for 1 to 2 minutes through a delivery end of surface area 0.5 cm² are recommended. If a relatively permanent blockade lasting weeks, months or even years is desired, direct currents in the range 0.4 to 6 mA for periods up to 1 hour or more are recommended. These parameters refer to the currents delivered to the nerve via a nerve cuff. As set out above, larger currents are required for delivery terminals not in contact with the nerve. It should be understood that these are exemplar parameters, and that other currents, durations and sizes of delivery ends could be used to achieve the desired blockade through ablation.

If or when nerve regeneration (recovery) and re-innervation causes the return of symptoms of unwanted or overactive nerve activity, typically weeks or months after the initial treatment, the nerve ablating treatment described in this invention can be repeated. This can be done whenever symptoms re-appear, similar to the case with repeated Botox injections, the difference being that in the case of the present invention, with an implanted conductor(s) or an implanted current source, no further significant cost is incurred. Another important difference is that the amount of attenuation in follow-up treatments can again be graded during the treatment itself. The amount of recovery of the normal function of the nerve resulting from such regeneration depends on the length between the lesion site and the target organ the axons in the nerve are required to regenerate in order to re-innervate the target organ (Fenrich & Gordon 2004). For example, if a nerve innervating the hand muscles is ablated close to the spinal cord, the length of nerve required to regenerate to reach the hand muscles is up to a meter, so the likelihood of many axons regenerating for this distance re-innervating on the muscle and restoring normal functioning of the hand is very low. On the other hand if the same nerve is severed close to the hand muscles, re-innervation can be quite successful, resulting in a restoration of useful hand function within months. Thus the duration of nerve blockade depends not only on the parameters of electrical current applied to the nerve to block or ablate it, but also on the site of this application.

The nerve ablation method and system of the invention provides a number of advantages. The method provides a means of reducing the activity of targeted nerves by controlled amounts. Unlike the pharmacological approaches discussed above, the reduction is immediate, and therefore the amount of reduction can be controlled by adjusting the level and duration of current. If and when recovery of nerve conduction occurs, the desired amount of reduction can be restored by controlling the external or internal current sources. No further surgery is required. The costs associated with regular, repeated injections of substances such as Botox are avoided.

When a Markush group or other grouping is used herein, all individual members of the group and all combinations and possible subcombinations of the group are intended to be individually included in the disclosure. Every formulation or combination of components described or exemplified herein can be used to practice the invention, unless otherwise stated. One of ordinary skill in the art will appreciate that methods, device elements, and materials other than those specifically exemplified can be employed in the practice of the invention without resort to undue experimentation. All art-known functional equivalents, of any such methods, device elements, and materials are intended to be included in this invention. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this invention. The invention illustratively described herein suitably may be practiced in the absence of any element or elements, limitation or limitations which is not specifically disclosed herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

Although the description herein contains many specificities, these should not be construed as limiting the scope of the invention, but as merely providing illustrations of some of the embodiments of the invention. Each reference cited herein is hereby incorporated by reference in its entirety. However, if any inconsistency arises between a cited reference and the present disclosure, the present disclosure takes precedence. Some references provided herein are incorporated by reference herein to provide details concerning the state of the art prior to the filing of this application, other references may be cited to provide additional or alternative device elements, additional or alternative materials, additional or alternative methods of analysis or application of the invention. It will be apparent to one skilled in the art that modifications may be made to the illustrated embodiment without departing from the scope of the invention as hereinafter defined in the claims.

### REFERENCES

Abdel-Gawad, M, Boyer, S, Sawan, M and Elhilali, M M. (2001) Reduction of bladder outlet resistance by selective stimulation of the ventral sacral root using high frequency blockade: a chronic study in spinal cord transected dogs. Journal of Urology 166:728-733.

Ade-Hall R A, and Moore A P (2000) Botulinum Toxin Type A in the treatment of lower limb spasticity in cerebral palsy. Cochrane Database Syst Rev, CD001408.

Amis A, Prochazka A, Short D, Trend P S and Ward A, (1987) Relative displacements in muscle and tendon during human arm movements. Journal of Physiology 389: 37-44.

Apkarian, J A and Naumann, S. (1991) Stretch reflex inhibition using electrical stimulation in normal subjects and subjects with spasticity. Journal of Biomedical Engineering 13:67-72.

Ashkan, K, Wallace, B, Bell, B A and Benabid, A L. (2004) Deep brain stimulation of the subthalamic nucleus in Parkinson's disease 1993-2003: where are we 10 years on? Br J Neurosurg 18: 19-34.

Benabid, A L, Pollak, P, Louveau, A, Henry, S and De Rougemont, J. (1987) Combined (thalamotomy and stimulation) stereotactic surgery of the VIM thalamic nucleus for bilateral Parkinson disease. Applied Neurophysiology 50:344-346.

Bhadra, N and Kilgore, K L (2004) Direct current electrical conduction block of peripheral nerve. IEEE Trans Neural Syst Rehabil Eng. 12, 313-324

Bhadra, N and Kilgore, K L. (2005) High-frequency electrical conduction block of mammalian peripheral motor nerve. Muscle Nerve 32:782-790.

Brindley, G S, Polkey, C E and Rushton, D. N. (1982) Sacral anterior root stimulators for bladder control in paraplegia. Paraplegia 20:365-381.

Broseta, J, Garcia-March, G, Sanchez-Ledesma, M J, Barbera, J and Gonzalez-Darder, J. (1987) High-frequency cervical spinal cord stimulation in spasticity and motor disorders. Acta Neurochir Suppl (Wien) 39:106-111.

Cattrell, M, Gerard, R W. (1935) The “inhibitory” effect of high-frequency stimulation and the excitation state of nerve. J. Physiol. 83, 407-415.

Fenrich K, and Gordon T. (2004) Canadian Association of Neuroscience Review: axonal regeneration in the peripheral and central nervous systems—current issues and advances. Can. J. of Neurol Science 31, 142-156.

Filali, M., Hutchison, W D, Palter, V N, Lozano, A M and Dostrovsky, J O. (2004) Stimulation-induced inhibition of neuronal firing in human subthalamic nucleus. Exp Brain Res 156(3):274-81.

Grill, W M, Jr. and Mortimer, J T. (1996) Quantification of recruitment properties of multiple contact cuff electrodes. IEEE Trans. Rehabil. Eng. (4(2):49-62.

Groen, J, and Bosch, J L. (2001) Neuromodulation techniques in the treatment of the overactive bladder. BJU Int 87(8):723-731.

Handa, Y, Yagi, R and Hoshimiya, N. (1998) Application of functional electrical stimulation to the paralyzed extremities. Neurologia Medico-Chirurgica 38:784-788.

Haugland, M & Sinkjaer, T. (1999) Interfacing the body's own sensing receptors into neural prosthesis devices. Technology & Health Care 7:393-399.

Horch, K W, and Dhillon, G S, ed. (2004) Neuroprosthetics. Theory and Practice. Vol. 2. World Scientific, New Jersey.

Jankovic, J, Cardoso, F, Grossman, R G, and Hamilton, W J., (1995) Outcome after stereotactic thalamotomy for parkinsonian, essential, and other types of tremor. Neurosurgery 37, 680-686; discussion 686-687.

Kirazli, Y, On, A Y, Kismali, B, and Aksit, R. (1998) Comparison of phenol block and botulinus toxin type A in the treatment of spastic foot after stroke: a randomized, double-blind trial. Am J Phys Med and Rehabil 77:510-515.

Kralj, A R, and Bajd, T. (1989) Functional Electrical Stimulation: Standing and Walking after Spinal Cord Injury. CRC Press, Boca Raton, Fla.

Landau, B, and Levy, R M. (1993) Neuromodulation techniques for medically refractory chronic pain. Annu Rev Med 44:279-287.

McCrea P H, Eng J J, and Willms R. (2004) Phenol reduces hypertonia and enhances strength; a longitudinal case study. Neurorehabil. Neural Repair 18, 112-116.

McCreery D B, Agnew W F, Yuen T G, and Bullara L. (1990) Charge density and charge per phase as cofactors in neural injury induced by electrical stimulation. IEEE Trans Biomed Eng 37, 996-1001.

Merrill D R, Bikson M, and Jefferys J G. (2005) Electrical stimulation of excitable tissue design of efficacious and safe protocols. J. Neurosci Methods 141:171-198.

Peckham P H, Marsolais, E B and Mortimer, J T (1980) Restoration of key grip and release in the C6 tetraplegic patient through functional electrical stimulation. J. Hand Surg. 5:462-469.

Peckham, P H, Keith, M W, Kilgore, K L, Grill, J H, Wuolle, K S, Thrope, G B, Gorman, P, Hobby, J, Mulcahey, M J, Carroll, S, Hentz, V R and Wiegner, A. Implantable Neuroprosthesis Research G (2001) Efficacy of an implanted neuroprosthesis for restoring hand grasp in tetraplegia: a multicenter study. Archives of Physical Medicine & Rehabilitation 82:1380-1388.

Prochazka, A, Gauthier, M, Wieler, M and Kenwell, Z. (1997) The bionic glove: an electrical stimulator garment that provides controlled grasp and hand opening in quadriplegia. Arch. Phys. Med. Rehabil. 78:608-614.

Scheiner A, Mortimer J T, and Roessmann U. (1990) Imbalanced biphasic electrical stimulation: muscle tissue damage. Ann Biomed Eng 18:407-425.

Shaker, H. and Hassouna, M M. (1999) Sacral root neuromodulation in the treatment of various voiding and storage problems. International Urogynecology Journal & Pelvic Floor Dysfunction 10:336-343.

Shaker, H S, Tu, L M, Robin, S, Arabi, K, Hassouna, M, Sawan, M and Elhilali, M M. (1998) Reduction of bladder outlet resistance by selective sacral root stimulation using high-frequency blockade in dogs: an acute study. J Urol 160(3 Pt 1):901-7.

Skold C, Levi R, and Seiger A. (1999) Spasticity after traumatic spinal cord injury: nature, severity, and location. Arch Phys Med Rehabil 80:1548-1557.

Solomonow, M, Eldred, E, Lyman, J and Foster, J. (1983) Control of muscle contractile force through indirect high-frequency stimulation. Am J Phys Med 62:71-82.

Strojnik, P, Acimovic, R, Vavken, E, Simic, V and Stanic, U. (1987) Treatment of drop foot using an implantable peroneal underknee stimulator. Scandanavian J. of Rehabil. Med. 19:37-43.

Tai, C, Roppolo, J R and de Groat, W C. (2004). Block of external urethral sphincter contraction by high frequency electrical stimulation of pudendal nerve. J Urol 172(5 Pt 1):2069-72.

Tai, C, Roppolo, J R and de Groat, W C (2005). Response of external urethral sphincter to high frequency biphasic electrical stimulation of pudendal nerve. J Urol 174(2):782-6.

Tai, C, Wang J, Wang X, de Groat W C, and Roppolo J R. (2007) Bladder inhibition or voiding induced by pudendal nerve stimulation in chronic spinal cord injured cats. Neurourol Urodynamics 26:570-577.

Vodovnik, L, Bowman, B R and Winchester, P. (1984) Effect of electrical stimulation on spasticity in hemiparetic patients. International Rehabilitation Medicine 6:153-156.

Vodovnik, L. (1981) Therapeutic effects of functional electrical stimulation of extremities. Medical and Biological Engineering & Computing 19:470-478.

Waltz, J. M. (1997) Spinal cord stimulation: a quarter century of development and investigation. A review of its development and effectiveness in 1,336 cases. Stereotactic & Functional Neurosurgery 69:288-299.

Ward A, Roberts G, Warner J, and Gillard S. (2005) Cost-effectiveness of botulinum toxin type a in the treatment of post-stoke spasticity. J Rehabil. Med. 37:252-257.

Whitwam J G, and Kidd C. (1975) The use of direct current to cause selective block of large fibers in peripheral nerves. Br J Anaesth 47:1123-1133.

Woo R. (2001) Spasticity: orthopedic perspective. J Child Neurol 16: 47-53.

Yu, D T, Chae, J, Walker, M E and Fang, Z P. (2001) Percutaneous intramuscular neuromuscular electric stimulation for the treatment of shoulder subluxation and pain in patients with chronic hemiplegia: a pilot study. Arch Phys Med Rehabil 82:20-25.

### PATENT DOCUMENTS

Heggeness, M H. Method and devices for intraosseous nerve ablation. U.S. Pat. No. 6,699,242, issued Mar. 2, 2004.

Kilgore, K L., Bhadra, N. Onset-Mitigating High Frequency Nerve Block. International Patent Application Publication No. WO 2009/058258, published May 7, 2009.

Nathan, R H. Device for generating hand function. U.S. Pat. No. 5,330,516, issued Jul. 19, 1994.

Prochazka, A, Wieler, M, Kenwell, Z R, Gauthier, M J A. (1996) Garment for applying controlled electrical stimulation to restore motor function. U.S. Pat. No. 5,562,707, issued Oct. 8, 1996.

Prochazka, A. Method and apparatus for controlling a device or process with vibrations generated by tooth clicks. International Patent Application Publication No. WO 2004/034937, published Oct. 16, 2003.

Sawan, M. and Elhilali, M M. Electronic stimulator implant for modulating and synchronizing bladder and sphincter function. U.S. Pat. No. 6,393,323, issued May 21, 2002.

All publications mentioned in this specification are indicative of the level of skill in the art to which this invention pertains. All publications are herein incorporated by reference to the same extent as if each individual publication was specifically and individually indicated to be incorporated by reference. Although the foregoing invention has been described in some detail by way of illustration and example, for purposes of clarity and understanding it will be understood that certain changes and modifications may be made without departing from the scope or spirit of the invention as defined by the following claims.

1. A method of treating a subject having unwanted or overactive nerve activity, comprising:
(a) applying one or more of direct current and charge imbalanced time varying current to a target nerve; and
(b) controlling the amplitude and the duration of the current such that there is a net charge delivered to the target nerve at a sufficient current density to cause controlled ablation of the target nerve until unwanted or overactive nerve activity is reduced in one or both of the target nerve and a target body tissue innervated by the target nerve.

2. The method of claim 1, wherein the current is direct current delivered to the target nerve at a current density of between 0.2 mA/cm² and 12 mA/cm².

3. The method of claim 2, wherein the current is applied for a duration of more than 10 seconds.

4. The method of claim 1, wherein the nerve is a peripheral nerve.

5. The method of claim 1, wherein the current is delivered while the subject is sedated or anesthetized.

6. The method of claim 1, wherein the current is delivered as charge imbalanced pulsatile current.

7. The method of claim 1, wherein the current is delivered to the nerve through an implanted conductor having a delivery portion positioned proximate to, or attached to, the target nerve.

8. The method of claim 7, wherein the current is delivered to the nerve with an implanted current source connected to the implanted conductor.

9. The method of claim 8, wherein the implanted current source is controlled by an external controller which is inductively coupled through the subject's skin to the implanted current source.

10. The method of claim 7, which further comprises, upon recovery of the ablated nerve and re-innervation of the target body tissue re-applying the current as in steps (a) and (b).

11. The method of claim 8, wherein the target nerve is one or more of a facial nerve, a spinal accessory nerve, a musculocutaneous nerve, a median nerve, a pudendal nerve, a sciatic nerve, a femoral nerve, and one or more branches of one of these target nerves.

12. A method of treating a subject having unwanted or overactive nerve activity, comprising:
(a) implanting an implant under the subject's skin, the implant including a passive electrical conductor having a pick-up portion and a delivery portion and being insulated between the pick-up portion and the delivery portion, the pick-up portion being configured to pick up at least a portion of a current flowing between a first surface electrode and a second surface electrode when positioned on the subject's skin, and to transmit the portion of the current to a target nerve;
(b) positioning the first surface electrode and the second surface electrode in spaced relationship on the subject's skin to make direct electrical contact with the subject's skin, with the first surface electrode positioned over the pick-up portion of the electrical conductor so the portion of the current is transmitted through the electrical conductor to the target nerve; and
(c) applying one or more of direct current and charge imbalanced time varying current between the first surface electrode and the second surface electrode to cause the portion of the electrical current to flow through the implant to be delivered to the target nerve; and
(d) controlling the amplitude and the duration of the current such that there is a net charge delivered to the target nerve at a sufficient current density to cause controlled ablation of the target nerve until unwanted or overactive nerve activity is reduced in one or both of the target nerve and a target body tissue innervated by the target nerve.

13. The method of claim 12, wherein the current is direct current delivered such that the portion of current which flows through the implant is at a current density of between 0.2 mA/cm² and 12 mA/cm².

14. The method of claim 13, wherein the current is applied for a duration of more than 10 seconds.

15. The method of claim 12, wherein the nerve is a peripheral nerve.

16. The method of claim 12, wherein the current is delivered while the subject is sedated or anesthetized.

17. The method of claim 12, wherein the current is delivered as charge imbalanced pulsatile current.

18. The method of claim 12, which further comprises, upon recovery of the target nerve and re-innervation of the target body tissue, re-applying the current as in steps (a) and (b).

19. The method of claim 16, wherein the target nerve is one or more of a facial nerve, a spinal accessory nerve, a musculocutaneous nerve, a median nerve, a pudendal nerve, a sciatic nerve, a femoral nerve, and one or more branches of one of these target nerves.

20. The method of claim 1, which further comprises providing a second implant beneath the subject's skin positioned to provide a return path for the current between the target nerve and the second surface electrode.

21. The method of claim 12, which further comprises providing a second implant beneath the subject's skin positioned to provide a return path for the electrical current between the target nerve and the second surface electrode.

22. The method of claim 1, wherein the implant is one implant from a plurality of implants, the method further comprising implanting each implant from the plurality of implants entirely under the subject's skin, each of the plurality of implants extending to a different target nerve, and positioning a plurality of surface electrodes on the subject's skin relative to the plurality of implants to cause nerve ablation to the different target nerves independently.

23. The method of claim 12, wherein the implant is one implant from a plurality of implants, the method further comprising implanting each implant from the plurality of implants entirely under the subject's skin, each of the plurality of implants extending to a different target nerve, and positioning a plurality of surface electrodes on the subject's skin relative to the plurality of implants to cause nerve ablation to the different target nerves independently.

24. A system for treating a subject, comprising:
an implant including an insulated electrical conductor having a delivery portion configured to deliver current to a target nerve; and
a current source configured to supply current in the form of one or more of direct current and charge imbalanced time varying current to the implant, and being configured to control the amplitude and the duration of the current such that a net charge may be delivered to the target nerve at a sufficient current density to cause controlled ablation of the target nerve.

25. The system of claim 24, wherein the delivery portion of the implant includes an electrical termination configured to deliver current to the target nerve at a current density of between 0.2 mA/cm² and 12 mA/cm².

26. The system of claim 25, wherein the current source is configured to be implanted beneath the subject's skin, and wherein the system further comprises an external controller configured to be inductively coupled through the subject's skin to the implanted current source in order to control the amplitude and the duration of the current delivered to the target nerve.

27. A system for treating a subject, comprising:
a first surface electrode and a second surface electrode configured to make electrical contact with the subject's skin and to transmit current to bodily tissue below the skin;
a stimulator electrically coupled to the first surface electrode and the second surface electrode, the stimulator being configured to supply current to the first surface electrode and the second surface electrode in the form of one or more of direct current and charge imbalanced time varying current, and the stimulator being configured with controls for the amplitude and the duration of the current such that a net charge may be delivered to a target nerve at a sufficient current density to cause controlled ablation of the target nerve;
an implant including a passive electrical conductor having a pick-up portion and a delivery portion and being insulated between the pick-up portion and the delivery portion, the pick-up portion being configured so that, once implanted beneath the skin, the pick-up portion picks up at least a portion of a current flowing between the first surface electrode and the second surface electrode when positioned on the subject's skin, and transmits the portion of the current to the target nerve.

28. The system of claim 27, wherein the stimulator is adapted to deliver direct current such that current is delivered to the target nerve at a current density of between 0.2 mA/cm² and 12 mA/cm², and wherein the delivery portion of the implant includes an electrical termination configured to deliver current to the target nerve at these current densities.

29. The system of claim 27, wherein the stimulator is adapted to deliver charge imbalanced pulsatile current to the target nerve.

30. The system of claim 28, wherein;
the pick-up portion forms an electrical termination having a sufficient surface area such that, once implanted in subcutaneous tissue below the first surface electrode, the portion of the current flows through the conductor, in preference to flowing through bodily tissue between the first surface electrode and the second surface electrode; and
the delivery portion forms an electrical termination to deliver the portion of the current to the target nerve, once implanted.

31. The system of claim 28, wherein:
the electrical termination at one or both of the pick-up portion and the delivery portion is formed from the uninsulated end of the conductor, or from other conductive or capacitive materials.

32. The system of claim 28, wherein the stimulator is configured to be external to the subject's body.

33. The system of claim 28, wherein at least the pick-up portion or the delivery portion is configured to include an enlarged surface area in the form of at least one of a coil, a spiral, a cuff, a rod, or a plate or a sheet in the form of an oval or a polygon.

34. The system of claim 28, wherein at least one of the pick-up portion or the delivery portion is formed from at least one of an uninsulated end of the conductor or from other conductive or capacitive materials.

35. The system of claim 28, further comprising:
an electrical return conductor having a collecting portion, a returning portion and an insulated portion between the collecting portion and the returning portion;
the collecting portion configured to collect a portion of the current delivered to the target nerve to return through the electrical return conductor in preference to returning through bodily tissue; and
the returning portion forming an electrical termination that returns the portion of the current to the second surface electrode via subcutaneous tissue and skin underlying the second surface electrode.

36. The system of claim 28, wherein the conductor is formed from at least one of a metal wire, carbon fibers, a conductive rubber or other conductive polymer, or a conductive salt solution in rubber.

37. The system of claim 28, wherein the first surface electrode and the second surface electrode include a conductive plate or a conductive sheet, a conductive gel electrode, a conductive rubber or polymer electrode that may be partially coated with an electrode paste or gel, or a moistened absorbent pad electrode.

38. The system of claim 28, further comprising a coating on one of both of the pick-up portion or the delivery portion, the coating being at least one of a conductive coating or capacitive coating, an oxide layer, an anti-inflammatory agent, an antibacterial agent, an antibiotic, or a tissue ingrowth promoter.

39. The system of claim 28, wherein the implant is one implant from a plurality of implants, each implant from the plurality of implants being configured to be implanted entirely under a subject's skin, each of the plurality of implants being of a sufficient length to extend to a different target nerve, the first surface electrode being one of a plurality of first surface electrodes and at least the first surface electrode and the second surface electrode being configured to be positioned relative to the plurality of implants to cause controlled ablation of a different target nerve independently.

40. The system of claim 28, wherein the pick-up portion is configured to pick up the portion of the electrical current via resistive coupling.

41. The system of claim 28, wherein the pick-up portion is configured to pick up the portion of the electrical current via capacitive and resistive coupling.
